(19) United States
(12) Patent Application Publication
(10) Pub. No.: US 2023/0017094 A1
(43) Pub. Date: Jan. 19, 2023

(54) DEVICES, METHODS AND ASSAYS FOR BIOLOGICAL MATERIALS

(71) Applicant: StemCell Technologies Canada Inc., Vancouver (CA)

(72) Inventors: Emanuel Nazareth, Vancouver (CA); Charis Segeritz-Walko, Vancouver (CA); Philipp Kramer, Vancouver (CA); Eric Jervis, Vancouver (CA); Colleen Umali, Vancouver (CA); Juan Hou, Vancouver (CA); Tyler Brown, Vancouver (CA); Nina Quiskamp, Vancouver (CA)

(21) Appl. No.: 17/779,830

(22) PCT Filed: Nov. 26, 2020
(86) PCT No.: PCT/CA2020/051616

§ 371 Date: May 25, 2022

Related U.S. Application Data
(60) Provisional application No. 62/940,493, filed on Nov. 26, 2019.

(51) Int. Cl. C12N 5/071; C12M 1/32; C12N 5/00

(52) U.S. Cl. CPC C12N 5/0677; C12M 23/12; C12N 5/0068; C12N 5/0688; C12N 2513/00; C12N 2533/90; C12N 2535/10; C12N 2537/00

(57) ABSTRACT

Described are devices for tethering biological materials, which in applicable embodiments support the growth and differentiation thereof. In a specific embodiment, the biological materials are cells and the cells grow/differentiate into tethered three-dimensional aggregates. The devices disclosed herein may be used in various methods/assays relating to tethered biological materials, such as to tethered three-dimensional aggregates of cells.

This application claims the benefit of United States Provisional patent application Ser. No. 62/940,493, filed Nov. 26, 2019, the entire content of which is incorporated herein by reference.

### TECHNICAL FIELD

This disclosure relates to the life sciences field. More particularly this disclosure relates to assays involving biological materials, said assays conducted using specialized devices.

### BACKGROUND

The culture of cells is an important aspect of investigating many life sciences hypotheses. Various approaches to culturing cells have been in use for greater than 100 years. In early days, cell culture relied on simple vessels using simple media formulations. While simple vessels and simple media formulations continue to be in widespread use, more specialized cell culture vessels and media formulations have emerged. Such specialized cell culture vessels and media formulations are largely aimed at enabling more complicated culture schemes and/or improving the efficiencies of more complicated culture schemes.

Multiwell plates, such as 6-well, 24-well, or 96-well plates, are widely available in the marketplace and may be used to culture the same or different types of cells in isolation from cells in other wells of the plate.

US Patent US20110086375 describes a cell culture plate for isolating within a common well, one or more cells from other cells in the same well. Such plates provide a wells-within-wells modality, sometimes referred to as microwells.

Azioune et al. (2009, Lab on a Chip, 9, 1640-1642) describe spot plates for localizing single cells on respective spots, and exposing the cells to permissive culture conditions.

There is a need for improved cell culture methods and devices that enable the simultaneous investigation of multiple conditions while also providing data to allow for conclusions to be made on the effect of the multiple conditions with statistical support.

### SUMMARY

The disclosure describes devices, methods, and assays for biological material(s). In a particular embodiment, biological material(s) are grown or assayed tethered to a device of the disclosure. In a more particular embodiment, the biological material(s) may be three-dimensional aggregates of cells, such as embryoid bodies (“EBs”), aggregates of pluripotent stem cells (“PSCs”) or differentiated PSCs, organoids or spheroids, or masses of chondrocytes.

In one broad aspect of this disclosure, anchorage surface devices for biological material(s) are provided. The anchorage surfaces comprise a plurality of microspots disposed on a first planar face. The first chemical attribute of the plurality of microspots is produced on exposing the first planar face to a source of energy. Each of the plurality of microspots having a first chemical attribute, separated by a pitch from a microspot adjacent thereto, and surrounded by a continuous interstitial surface having a second chemical attribute, the second chemical attribute being different than the first chemical attribute, wherein the first chemical attribute supports the tethering of biological materials thereto and the second chemical attribute is not supportive of tethering biological materials thereto.

In one embodiment, the pitch is between about 20 μm and 5000 μm, and preferably between about 500 μm and 1500 μm.

In one embodiment, the plurality of microspots have the same shape. In a specific embodiment, the plurality of microspots are circular or elliptical, triangular, or quadrilateral.

In one embodiment, a spacing between two adjacent microspots taken from a first edge of a first microspot to a closest first edge of an adjacent second microspot is between about 30 μm and 3000 μm.

In one embodiment, said spacing is preferably between about 100 μm and 1000 μm.

In one embodiment, anchorage surface may further comprise up to 10 microspots, up to 50 microspots, up to 100 microspots, up to 250 microspots, up to 500 microspots, up to 1000 microspots, or more.

In one embodiment, anchorage surface may further comprise a leak-proof physical barrier attached to the first face, the physical barrier circumscribing the plurality of microspots. In one embodiment the physical barrier defines at least one side wall and an open top end. In one embodiment, anchorage surface may further comprise more than one physical barrier attached to the first face, each of the more than one physical barrier circumscribing each of the plurality of microspots or a respective set of plurality of microspots. In one embodiment, the more than one physical barrier is comprised in a well-defining member.

In one embodiment, anchorage surface may further comprise a fluid coating supplement.

In one embodiment, the fluid coating supplement is preferentially engaged with the plurality of microspots in comparison to the continuous interstitial surface. In one embodiment, the fluid coating supplement is a solution of one or more extracellular matrix proteins. In one embodiment, the one or more extracellular matrix proteins include one or more of fibronectin, collagen, laminin, elastin, vitronectin, entactin, heparin sulphate, or proteoglycan. In one embodiment, the fluid coating supplement is Matrigel

In one embodiment, the first chemical attribute is relatively more hydrophilic than the second chemical attribute. In one embodiment, the first chemical attribute is hydrophilic and the second chemical attribute is hydrophobic.

In one embodiment, the first chemical attribute carries a charge different from the second chemical attribute.

In one embodiment, the first chemical attribute carries a functional group different from the second chemical attribute. In one embodiment, the functional group of the first chemical attribute is a carboxyl group.

In another aspect, this disclosure provides for use(s) of the anchorage surface according to any of the foregoing for growing a three-dimensional aggregate of cells

In another broad aspect of the disclosure, methods of growing a three-dimensional aggregate of cells are provided. The methods comprise: providing an anchorage surface having a plurality of microspots disposed on a first planar face thereof, each of the plurality of microspots having a first chemical attribute, separated by a pitch from a microspot adjacent thereto, and surrounded by a continuous interstitial surface having a second chemical attribute, the second chemical attribute being different than the first chemical attribute; contacting the plurality of microspots with a cell suspension; and culturing one or more anchorage-dependent cells of the cell suspension in a supportive culture medium under supportive culture conditions, wherein the first chemical attribute supports growth of the one or more anchorage-dependent cells to yield the three-dimensional aggregate of cells tethered to a microspot and the second chemical attribute does not support growth of the one or more anchorage-dependent cells to yield the three-dimensional aggregate of cells (e.g. the three-dimensional aggregate of cells is tethered to a microspot and the continuous interstitial surface does not support anchorage-dependent growth of the cell suspension).

In one embodiment, the methods further comprise contacting the plurality of microspots with a fluid coating supplement for a time sufficient to allow some or all of the fluid coating supplement to bind the plurality of microspots before contacting the plurality of microspots with the cell suspension, and optionally removing excess fluid coating supplement.

In one embodiment, the supportive culture medium includes a dilution of the fluid coating supplement. In one embodiment, the supportive culture medium includes a sub-gelation dilution of the fluid coating supplement.

In one embodiment, the fluid coating supplement is a solution of one or more extracellular matrix proteins. In one embodiment, the one or more extracellular matrix proteins include one or more of fibronectin, collagen, laminin, elastin, vitronectin, entactin, heparin sulphate, or proteoglycan. In one embodiment, the fluid coating supplement is Matrigel.

In one embodiment, the cell suspension is a suspension of pluripotent stem cells (“PSC”), a suspension of PSC-derived cells, or a suspension of primary cells. In one embodiment, the suspension of primary cells is a dissociated tissue sample or a blood sample. In one embodiment, the dissociated tissue sample is derived from an epithelial tissue. In one embodiment, the epithelial tissue includes at least one stem or progenitor cell. In one embodiment, the epithelial tissue is at least a portion of a lung, a kidney, a pancreas, a liver, a small intestine, a large intestine, a stomach, a prostate, or mammary.

In one embodiment, the three-dimensional aggregate of cells is an embryoid body, an aggregate of PSC, an organoid, or a mass of chondrocytes.

In embodiments where the three-dimensional aggregates of cells is an organoid, the organoid may be lumenized and/or polarized. In one embodiment, the organoid is one of: a lung organoid; a kidney organoid; a pancreatic organoid; a liver organoid; a small intestinal, including an duodenal, an ileal, an jejunal organoid; a large intestinal, including an organoid derived using cells from the cecum, ascending, transverse or descending colon, organoid; a stomach organoid, including an antral or fundal organoid; a prostate organoid; or a mammary organoid.

In one embodiment, reduced off-target cell differentiation occurs between or about a first three-dimensional aggregate of cells and an adjacent second three-dimensional aggregate of cells compared to adjacent first and second three-dimensional aggregates of cells not grown using the anchorage surface.

In one embodiment, the one or more anchorage-dependent cells are primary kidney stem or progenitor cells or PSC-derived kidney stem or progenitor cells and the three-dimensional aggregates of cells are kidney organoids. In one embodiment, a first of the kidney organoids and an adjacent second of the kidney organoids contain fewer neuroectodermal cells and/or stromal cells about its periphery or therebetween compared to adjacent first and second kidney organoids not grown using the anchorage surface.

In one embodiment, the one or more anchorage dependent cells are mesenchymal stem cells or PSC-derived mesenchymal stem cells and the three-dimensional aggregate of cells is a mass of chondrocytes.

In one embodiment, the methods may further comprise exposing a first three-dimensional aggregate of cells to a first condition and exposing a second three-dimensional aggregate of cells to a second condition. In one embodiment, the first condition is a test condition and the second condition is a control condition. In one embodiment, effects of the first condition and the second condition are monitored over a period of time.

In one embodiment, the methods may further comprise monitoring effects of the first condition on the first three-dimensional aggregate of cells and a second condition on the second three-dimensional aggregate of cells. In one embodiment, the first condition is a test condition and the second condition is a control condition. In one embodiment, effects of the first condition and the second condition are monitored over a period of time.

In another broad aspect of the disclosure, assays using a three dimensional aggregate of cells grown (and tethered to an anchorage surface) in accordance with the foregoing devices and methods are provided.

In another broad aspect of the disclosure, three-dimensional aggregates of cells grown (and tethered to an anchorage surface) in accordance with the foregoing devices and methods are provided.

Other features and advantages of the present invention will become apparent from the following detailed description. It should be understood, however, that the detailed description and the specific examples while indicating preferred embodiments of the invention(s) are given by way of illustration only, since various changes and modifications within the spirit and scope of the invention will become apparent to those skilled in the art from this detailed description.

### BRIEF DESCRIPTION OF THE DRAWINGS

Embodiments of the application will now be described in greater detail with reference to the attached figures in which:

FIG. 1 is a perspective view of one embodiment of an anchorage surface device as disclosed herein.

FIG. 2 is a top plan view of one embodiment of an anchorage surface device as disclosed herein.

For simplicity, only two anchorage surfaces are shown, but it is possible that others could be included on anchorage surface device.

FIG. 3 is a sectional view of one embodiment of an anchorage surface device taken through the line A of FIG. 2.

FIG. 4 is a top plan view of one embodiment of an anchorage surface comprising a plurality of microspots as disclosed herein. As in FIG. 2, only two anchorage surfaces are shown, but it is possible that others could be included on anchorage surface device.

FIG. 5 is an exploded perspective view of one embodiment of an anchorage surface device further comprising a well-defining member.

FIG. 6 depicts an anchorage surface (within a receptacle of an anchorage surface device) comprising a plurality of microspots having growing A540 cancer cells anchored thereto. A representative image using a fluorescent microscope of A540 cells stained with Hoechst dye taken at 2X magnification (A). A phase contrast image corresponding to the image of FIG. 6A (B).

FIG. 7 depicts confocal imaging results of differentiated WLS-1C iPSC-derived kidney aggregates (organoids) using the STEMdiff™ Kidney Organoid Kit. Approximately 3000-7000 single H9 (WA09) ES cells were seeded per well. Panels A) to E) show the aggregates anchored to a microspot of an anchorage surface device as disclosed herein. Panels F) to J) show the aggregates formed within a well of a standard 96-well plate (Costar). Panels B) and G) show staining for lotus tetragonolobus lectin (LTL), a proximal tubule marker. Panels C) and H) show staining for beta III-tubulin (TUJ1), a neural ectoderm marker. Panels D) and I) show staining for vimentin (VIM), a mesenchymal marker. Panels E) and J) show DAPI staining. Panels A) and F) show a merge. The scale bar represents 200 μm.

FIG. 8 depicts PSC-derived kidney aggregates (organoids) seeded in a well of standard 96-well plate (A) or in a well of a 96-well plate comprising a plurality of microspots disposed on the well bottom (B). Approximately 1000-11000 single H9 or 1C cells were seeded per well and differentiated using the STEMdiff™ Kidney Organoid Kit. The scale bar represents 1 mm.

FIG. 9 depicts hPSC-derived kidney aggregates (organoids) anchored respectively to a plurality of microspots. The hPSC-derived aggregates are stained with lotus tetragonolobus lectin or LTL (A), podocalyxin or PODXL (B), and E-cadherin or ECAD (C). FIG. 9D depicts a composite of the images in panels (A)-(C). FIG. 9E depicts a binary mask in which a pixel collocates any positive signal from panels (A)-(C).

FIG. 10 shows the results of an image analysis algorithm performed on the kidney aggregates depicted in FIG. 9. Two different aggregates (as demarcated by the dashed box) are assessed for intensity of LTL (A), PODXL (B), and ECAD (C). Intensity data along with an average pixilation score for each aggregate is summarized (D).

FIG. 11 shows a regression analysis comparing automated and manual counting of tethered aggregates. Each dot corresponds to the number of aggregates in a single receptacle of a 96-well anchorage surface device. A linear fit between the two methods of counting is observed, with an R² of 0.81.

FIG. 12 depicts live cell images taken on day 7 of aggregates having grown anchored to microspots in respective wells of an anchorage surface device. The upper right region bounded by a solid-lined white box corresponds to tethered aggregates having grown out of seeded cell fragments; otherwise, single cells obtained from a tissue sample were seeded. Mouse liver progenitor aggregates were generated in the top row. Human colonic aggregates are depicted in the second row from the top. Human small intestinal aggregates are depicted in the center row. Mouse prostate aggregates are depicted in the second row from the bottom. Human pulmonary aggregates are depicted in the bottom row. Each representation of a receptacle (well bottom) consists of 25 fields at 10× magnification that were stitched together (scale: well diameter is 6.35 mm).

FIG. 13 depicts a time course of aggregate formation as tethered to respective microspots of the anchorage surface device of FIG. 12. The images in panels A) through F) correspond to the indicated time point at the indicated magnification. Panels G) through L) zoom in on the receptacle (well bottom) demarcated by a solid-lined box in panels A) through F). Panels M) through R) zoom in on the single anchorage surface (microspot) of a receptacle (well bottom) as demarcated by a solid-lined box in panels G) through L). White arrows in N demarcate lumenization. The scale bar represents 250 μm.

FIG. 14 depicts formation of tethered mouse liver progenitor aggregates (organoids) derived from single cells/fragments of adult mouse liver biopsies at day 7 under different cell seeding densities and Corning® Matrigel® concentrations. Cell or fragment numbers seeded into a receptacle of an anchorage surface device were varied as indicated in panels A) through L). Panels A)-C) and G)-I) correspond to an overlay of undiluted Matrigel (100%), and panels D)-F) and J)-L) correspond to an overlay of culture medium including 5% Matrigel.

FIG. 15 depicts tissue-derived adult mouse liver progenitor tethered aggregates (organoids) at day 7 formed after seeding of 50,000 cells in culture medium including 5% Matrigel. Mouse liver aggregates were stained with Hoechst (A), and antibodies against HNF4a (B), ZO1 (C), Ezrin (D). A phase contrast image of the same receptacle (well) bottom is shown in panel (E). The scale bar represents 500 μm.

FIG. 16 depicts an analysis of the mouse liver aggregates (organoid) depicted in FIG. 15. Panel A) depicts an overlay of HNF4a, Ezrin and Z01 staining. Panel B) depicts, at an increased magnification (the scale bar represents 100 μm), the aggregate demarcated by the box in panel A). The distribution of equivalent diameter of all formed mouse liver aggregates (organoids) in the receptacle (well) is shown in panel C), where the dot represents where the aggregate (organoid) of panel B) lies in the distribution.

FIG. 17 depicts confocal microscopy of different Hoechst stained sections of day 7 mouse liver aggregates (organoid) along different focal planes. The mouse liver aggregates (organoid) were formed from 50,000 seeded single cells in culture medium including 5% Matrigel. Panel A) shows images at the indicated scales of a first section taken across three different focal planes: XY, XZ, and YZ. Panel B) shows images at the indicated scales of a second section taken across three different focal planes: XY, XZ, and YZ. Flattening of the aggregates arises as the cultures are fixed for microscopy.

FIG. 18 depicts tissue-derived human (large) intestinal aggregates (organoids) at day 7 under different seeding densities, Matrigel concentrations, and either dissociated into single cells or cell fragments. Single cell or cell fragment numbers seeded into a receptacle of an anchorage surface device were varied as indicated in panels A) through L). Panels A)-C) and G)-I) correspond to an overlay of undiluted Matrigel (100%) before adding culture medium, and panels D)-F) and J)-L) correspond to culture medium including 5% Matrigel. Scale: well diameter is 6.35 mm.

FIG. 19 depicts staining of day 7 tissue-derived human (large) intestinal tethered aggregates (organoids) formed from 50,000 cells in culture medium including 5% Matrigel. Human (large) intestinal aggregates (organoids) were stained with Hoechst (A), and antibodies against Muc2 (B), Villin (C), EpCAM (D). A phase contrast image of the same receptacle (well) bottom is shown in panel (E). The scale bar represents 500 μm.

FIG. 20 depicts confocal microscopy of different Hoechst stained sections of day 7 human (large) intestinal tethered aggregates (organoid) along different focal planes. The human (large) intestinal aggregates (organoids) were formed from 50,000 seeded single cells in culture medium including 5% Matrigel. Panel A) shows images at the indicated scales of a first section taken across three different focal planes: XY, XZ, and YZ. Panel B) shows images at the indicated scales of a second section taken across three different focal planes: XY, XZ, and YZ. Flattening of the aggregates arises as the cultures are fixed for microscopy.

FIG. 21 depicts tissue-derived human small intestinal tethered aggregates (organoids) at day 7 under different seeding densities, Matrigel concentrations, and either dissociated into single cells or cell fragments. Cell or fragment numbers seeded into a receptacle of an anchorage surface device were varied as indicated in panels A) through L). Panels A)-C) and G)-I) correspond to an overlay of undiluted Matrigel (100%) before adding culture medium, and panels D)-F) and J)-L) correspond to culture medium including 5% Matrigel.

FIG. 22 depicts staining of day 7 tissue-derived human small intestinal tethered aggregates (organoids) formed from 50,000 cells in culture medium including 5% Matrigel. Human small intestinal aggregates were stained with Hoechst (A), and antibodies against Muc2 (B), Villin (C), EpCAM (D). The scale bar represents 500 μm.

FIG. 23 depicts confocal microscopy of different Hoechst stained sections of day 7 human small intestinal tethered aggregates (organoid) along different focal planes. The human small intestinal aggregates (organoid) were formed from 50,000 seeded single cells in culture medium including 5% Matrigel. Panel A) shows images at the indicated scales of a first section taken across three different focal planes: XY, XZ, and YZ. Panel B) shows images at the indicated scales of a second section taken across three different focal planes: XY, XZ, and YZ. Flattening of the aggregates arises as the cultures are fixed for microscopy.

FIG. 24 depicts tissue-derived mouse prostate tethered aggregates (organoids) at day 7 under different seeding densities, Matrigel concentrations, and either dissociated into single cells or cell fragments. Cell numbers seeded into a receptacle of an anchorage surface device were varied as indicated in panels A) through L). Panels A)-F) correspond to an overlay of undiluted Matrigel (100%) before adding culture medium, and panels G)-L) correspond to culture medium including 5% Matrigel.

FIG. 25 depicts staining of day 7 mouse prostate tethered aggregates (organoids) formed from 25,000 mouse prostate cells in culture medium including 5% Matrigel. Mouse prostate aggregates were stained with Hoechst (A), and antibodies against Keratin 8 (B) and Keratin 14 (C). The scale bar represents 500 μm.

FIG. 26 depicts confocal microscopy of different Hoechst stained sections of day 7 mouse prostate tethered aggregates (organoid) along different focal planes. The mouse prostate aggregates (organoid) were formed from 55,000 seeded single cells with a 100% Matrigel overlay before adding culture medium. Panel A) shows images at the indicated scales of a first section taken across three different focal planes: XY, XZ, and YZ. Panel B) shows images at the indicated scales of a second section taken across three different focal planes: XY, XZ, and YZ. Flattening of the aggregates arises as the cultures are fixed for microscopy.

FIG. 27 depicts tissue-derived human pulmonary tethered aggregates (organoids) at day 7 under different seeding densities and Matrigel concentrations. Cell numbers seeded into a receptacle of an anchorage surface device were varied as indicated in panels A) through L). Panels A)-D) correspond to no overlay of Matrigel (0%), panels E)-H) correspond to culture medium including 5% Matrigel diluted in culture medium, and panels I)-L) correspond to an overlay of undiluted Matrigel (100%) before adding culture medium.

FIG. 28 depicts staining of day 7 tissue-derived human pulmonary tethered aggregates (organoids) formed from 40,000 human airway cells with no Matrigel overlay. Human pulmonary aggregates were stained with Hoechst (A), and antibodies against CD271 (B), AcTub (C), and CD49f (D). The scale bar represents 500 μm.

FIG. 29 depicts confocal microscopy of different Hoechst stained sections of day 7 human pulmonary tethered aggregates (organoid) along different focal planes. The human pulmonary aggregates (organoid) were formed from 20,000 seeded single cells with a 100% Matrigel overlay. Panel A) shows images at the indicated scales of a first section taken across three different focal planes: XY, XZ, and YZ. Panel B) shows images at the indicated scales of a second section taken across three different focal planes: XY, XZ, and YZ. Flattening of the aggregates arises as the cultures are prepared for microscopy.

FIG. 30 depicts staining of day 7 tissue-derived human pulmonary tethered aggregates (organoids) formed from 40,000 cells with a no Matrigel overlay, in culture medium including 5% Matrigel, and a 100% Matrigel overlay. Human pulmonary aggregates were stained with Hoechst, and antibodies against CD271, AcTub, and Keratin 14. Shown is the composite of these 4 channels. The scale bar represents 500 μm.

FIG. 31 depicts formation of day 14 tethered pancreatic duct aggregates (organoids) formed from human pancreatic progenitor cells under differing conditions, as described herein. Cell numbers seeded into a receptacle of an anchorage surface device were varied as indicated in panels A) through 0). Panels A)-E) correspond to no overlay of Matrigel (0%), panels F)-J) correspond to culture medium including 1% Matrigel, and panels K)-0) correspond to culture medium including 5% Matrigel. The scale bar represents 500 μm.

FIG. 32 depicts formation of day 14 tethered pancreatic duct aggregates (organoids) formed from 60,000 human pancreatic progenitor cells seeded into a receptacle of an anchorage surface device. Panels A)-C) are whole well images, and panes D)-F) are regions of the same panels enlarged to show morphology. Panels A) and D) correspond to no overlay of Matrigel (0%), panels B) and E) correspond to culture medium including 1% Matrigel, and panels C) and F) correspond to culture medium including 5% Matrigel. The scale bar represents 500 μm.

FIG. 33 depicts formation of day 7 tethered pancreatic duct aggregates (organoids) formed from 60,000 human pancreatic progenitor cells seeded into a receptacle of an anchorage surface device. Panels A)-C) are whole well images, and panes D)-F) are regions of the same panels enlarged to show morphology. Panels A) and D) correspond to no overlay of Matrigel (0%), panels B) and E) correspond to culture medium including 1% Matrigel, and panels C) and F) correspond to culture medium including 5% Matrigel. The scale bar represents 500 μm.

FIG. 34 depicts formation of day 28 tethered human airway aggregates (organoids) formed from human bronchial epithelial cells (HBEC) under differing conditions, as described herein. Cell numbers seeded into a receptacle of an anchorage surface device were varied as indicated in panels A) through H). Panels A)-D) correspond to no overlay of Matrigel (0%), and panels E)-H) correspond to culture medium including 5% Matrigel. Panels A), B), E), and F) correspond to cells being cultured in PneumaCult™ AOK Seeding Medium for the initial 4 days, and panels C), D), G), H) correspond to cells being cultured in seeding media for the initial 7 days. Thereafter, PneumaCult™ AOK Seeding Differentiation Medium was used for the duration of the assay. The scale bar represents 500 μm.

FIG. 35 depicts the effects of treatment with amiloride, forskolin, and genistein (AFG) on day 28 tethered human airway aggregates (organoids). The aggregates were formed from 40,000 human bronchial epithelial cells (HBEC) seeded into a receptacle of an anchorage surface device and cultured in cultured medium including 5% Matrigel. HBECs were cultured for the initial 7 days in PneumaCult™ AOK Seeding Medium and thereafter in PneumaCult™ AOK Differentiation Medium for the duration of the experiment. Panel A) corresponds to the well immediately prior to addition of AFG (T=Oh), and panel B) corresponds to the same well 6 h after AFG addition. Panel C) corresponds to the well immediately prior to addition of DMSO (T=Oh), and panel D) corresponds to the same well 6 h after DMSO addition. The AFG treated aggregate demarcated by the box in panel A) corresponds to the same aggregate demarcated by the box in panel B). Similarly, the DMSO treated aggregate demarcated by the box in panel C) corresponds to the same aggregate demarcated by the box in panel D). The scale bar represents 500 μm.

FIG. 36 depicts the effects after 6 hours of treatment with amiloride, forskolin, and genistein (AFG) (panels A, B, C) in comparison to a DMSO control (panels D, E, F) on day 28 tethered human airway aggregates (organoid). The aggregates were formed from 40,000 human bronchial epithelial cell (HBEC) seeded into a receptacle of an anchorage surface device and cultured in cultured medium including 5% Matrigel. HBECs were cultured for the initial 7 days in PneumaCult™ AOK Seeding Medium and thereafter in PneumaCult™ AOK Differentiation Medium for the duration of the experiment. Panels A) and D) correspond to phase contrast images of an HBEC-derived aggregate, panels B) and E) correspond to the same aggregates of panels A) and D), respectively, co-stained with Calcein AM (live-cell marker), and panels C) and F) correspond to the same aggregates of panels A) and B) and panels D) and E), respectively, co-stained with ethidium homodimer 1 stain (ETHD, dead-cell marker). The scale bar represents 200 μm.

FIG. 37 depicts the quantification of swollen lumen area after treatment with amiloride, forskolin, and genistein (AFG) on day 28 tethered human airway aggregates (organoids). The aggregates were formed as in FIG. 35. Panel A) corresponds to an organoid prior to AFG addition (T=Oh). Panel B) corresponds to the same organoid image post analysis, with lumens outlined and numbered 1 to 4. Panel C) corresponds to an organoid 6 h after AFG addition. Panel D) corresponds to the same organoid image post analysis, with lumens outlined and numbered 1 to 4. Geometric properties of each lumen can be obtained, such as area and circularity. Area, % Area change, and other geometric properties of lumens are summarized in Panel E). IMAGEJ was used to median filter and minimum filter before applying thresholds to mask the lumens. The scale bar represents 500 μm.

FIG. 38 depicts formation of tethered human airway aggregates (organoids) from human bronchial epithelial cells (HBEC) at day 5, day 12, and day 28 under different conditions. 30,000 cells were seeded into a receptacle of an anchorage surface device. Panels A)-C) correspond to no overlay of Matrigel (0%), and panels D)-F) correspond to culture medium including 5% Matrigel. Panels A) and D) correspond to the aggregate at day 5, panels B) and E) correspond to the aggregate at day 12, and panels C) and F) correspond to the aggregate at day 28. Panels A) to C) correspond to the same aggregate at different time points, and panel D) to F) correspond to the same aggregate at different time points. The scale bar represents 250 μm.

FIG. 39 depicts confocal images of staining of day 28 tethered human airway aggregates (organoids) from HBECs cultured in 5% Matrigel. Human airway aggregates were stained with DAPI (A), and antibodies against MUCSAC (B), and AcTub (C). A composite image is shown in panel (D). The scale bar represents 100 μm.

### DETAILED DESCRIPTION

This disclosure describes anchorage surface devices for growing three-dimensional aggregates out of one or more anchorage-dependent cells, methods of manufacturing such anchorage surface devices, and methods of growing three-dimensional aggregates using the foregoing devices.

### Device

In a first aspect of this disclosure are provided anchorage surface devices 1 for biological material(s). Anchorage surface device 1 is not constrained by size, shape, or material, provided it is capable of tethering one or more biological material(s) thereto (and maintaining such tethering). In embodiments where the biological material corresponds to cells, cells that are tethered to the anchorage surface device may expand, or retain the potential to expand. A typical biological material for use with anchorage surface device 1 may include a tissue or a tissue fragment, a cell or an aggregates of cells, an organelle or a vesicle (such as an extracellular vesicle or an exosomes), a protein, or a nucleic acid. Regardless, the person skilled in the art will know how to obtain such biological materials for use with anchorage surface device 1, or the methods/assays, disclosed herein.

In one embodiment the biological material is one or more cells, such as may be comprised in a cell suspension used to contact anchorage surface device 1. In a specific embodiment the one or more cells, as may be comprised in a cell suspension, are anchorage-dependent cells (i.e. cells that grow as an adhered culture).

In one embodiment the biological material is an aggregate of cells, or a plurality of cell aggregates respectively, tethered to the anchorage surface device, such as may grow out of a cell suspension that includes one or more anchorage-dependent cells. In one embodiment the aggregate(s) of cells is a three-dimensional aggregate that remains tethered to the anchorage surface device as it grows. Non-limiting examples of three-dimensional aggregates include an embryoid body (formed from pluripotent stem cells (“PSC”), such as embryonic stem cells (“ESC”) or induced pluripotent stem cells (“iPSC”), an aggregate of undifferentiated PSC, an organoid, a spheroid, or a mass of chondrocytes (such as may be formed following a chondrogenesis assay).

Anchorage surface device 1 may be made of any material that is compatible with biological materials. In preferred embodiments, anchorage surface device 1 is made using readily accessible and cost non-prohibitive material(s). In one embodiment, anchorage surface device 1 comprises glass. In one embodiment, anchorage surface device 1 comprises a polymer. In one embodiment, anchorage surface device 1 comprises both glass and a polymer. In one embodiment, the polymer is polyethylene glycol (“PEG”) or is PEG-based. In one embodiment, anchorage surface device 1 is stratified. In a specific such embodiment, anchorage surface device 1 comprises a polymer top layer overlying a glass bottom.

Anchorage surface device 1 may assume any size or shape that is suitable for tethering biological material(s) thereto. Anchorage surface device 1 may be a substantially planar surface or may include one or more sidewall. Anchorage surface device 1 may include one or more physical barriers (such as one or more sidewall) that divide anchorage surface device 1 into a plurality of separated receptacles. In one embodiment, anchorage surface 1 may be approximately the size and shape of a standard microscope slide. In one embodiment, anchorage surface 1 may be approximately the size and shape of a standard petri dish, or a differently-sized circular dish such as a 35 mm dish. In one embodiment, anchorage surface device 1 may be approximately the size and shape of a cell culture plate or flask. In one embodiment, the cell culture plate is a multiwell plate (i.e. comprises a plurality of wells), such as a 6-well plate, a 12-well plate, a 24-well plate, a 96-well plate, or a 384-well plate. In a preferred embodiment, anchorage surface device 1 is a 96-well plate.

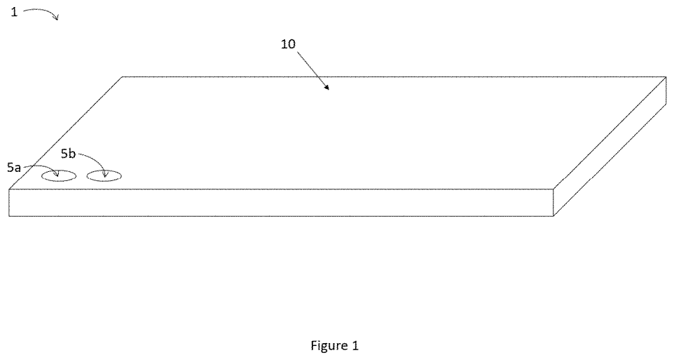

An embodiment of anchorage surface device 1 is shown in FIG. 1, wherein only 2 of n anchorage surfaces 5 (i.e. 5a and 5b) are depicted. Although a majority of the following description is in the context of a device 1 comprising more than one anchorage surface 5, it may be equally applicable to embodiments of device 1 that only include one anchorage surface 5.

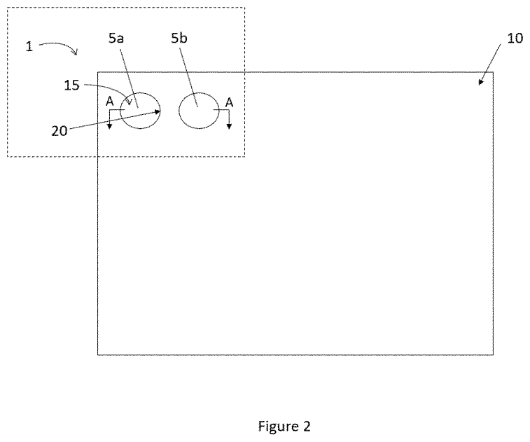

Anchorage surfaces 5 may be disposed on the same plane as a first planar face 10 of device 1, or may be recessed relative to first planar face 10. In one embodiment where anchorage surfaces 5 are disposed on the same plane as first planar face 10 they may be surrounded by a physical barrier (not shown). Regardless of whether anchorage surfaces 5 are recessed relative to first planar face 10 or are disposed on first planar face 10 and then surrounded by one or more physical barrier, the end result is a receptacle 15 surrounded by one or more sidewall 20 (FIG. 2). One or more physical barrier may be desirable where biological materials tethered to anchorage surface device 1 are contacted with different experimental conditions or where biological materials are contacted with identical and/or replicate experimental conditions, or both.

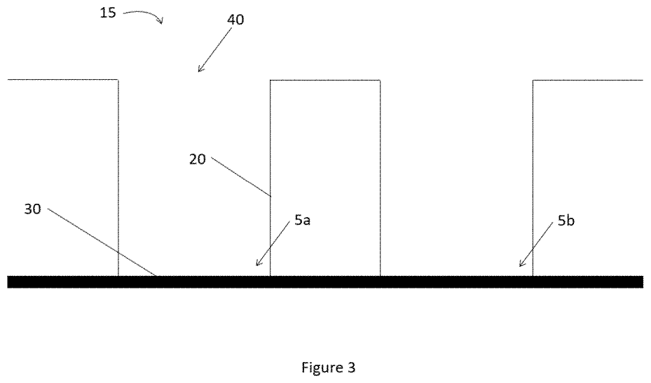

In embodiments where anchorage surface device 1 is comprised in a multi-well plate, each of the plurality of wells corresponds to receptacle 15 (FIG. 2). In such embodiments, each receptacle 15 is defined by a closed bottom wall 30 (which may correspond with or underlie anchorage surface 5), an open top end 40 and one or more side wall 20 extending from bottom wall 30 to open top end 40 (FIG. 3).

It is important that if closed bottom wall 30 is not integral with side wall 20, that these components are attached in such a way so as to prevent leakage of any fluid that may be contained within receptacle 15. Any type of attachment means are contemplated herein, such as adhesives, cements, or welding.

Each receptacle 15 may be any shape or hold any volume, which will depend on the particular application for which device 1 is used. Bottom wall 30 is preferably flat or substantially flat. A flat or substantially flat bottom wall should reduce the likelihood of uneven distribution of biological materials that are deposited into receptacle 15 and/or onto first planar face 10.

In one embodiment, side wall 20 may be substantially vertical (or orthogonal to bottom wall 30) (FIG. 3). In one embodiment, side wall 20 may be substantially vertical (or orthogonal to bottom wall 30) at an upper portion thereof but curve inwardly at a lower portion thereof. In one embodiment, an upper portion of side wall 20 is substantially vertical (or orthogonal to bottom wall 30) and is connected to bottom wall 30 by an angled wall member. In such an embodiment an area of bottom wall 30 is smaller than an area of open top end 40.

In one embodiment, receptacle 15 is substantially cylindrically shaped. In one embodiment, side wall 20 height may be approximately 0.5 cm, approximately 1 cm, approximately 1.5 cm or approximately 2 cm. In one embodiment, a diametric width of bottom wall 30 may be approximately 0.5 cm, approximately 1 cm, approximately 2 cm, approximately 3 cm, approximately 4 cm, approximately 5 cm, or more. In one embodiment, sidewall 20 may be curved.

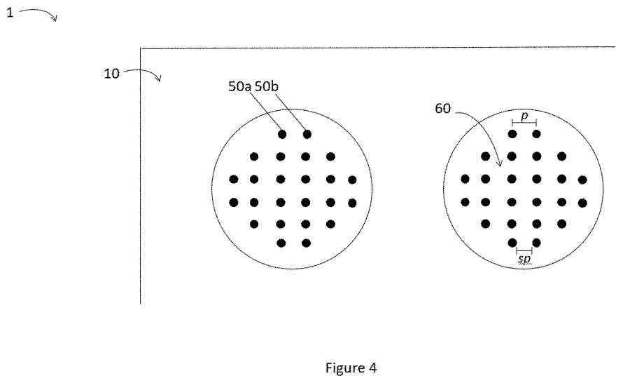

In the embodiment depicted in FIG. 3, bottom wall 30 (or first planar face 10) includes a plurality of microspots 50 (and 50a, 50b, 50c, and so on when referred to individually), which plurality of microspots may be seen in FIG. 4. In the context of receptacle 15, plurality of microspots 50 face open top end 40. Here, it is important to clarify the relationship between anchorage surfaces 5 and plurality of microspots 50.

In one embodiment, anchorage surface 5 may correspond to a microspot, and such anchorage surface or microspot may be continuous from an edge to edge thereof. In such an embodiment each anchorage surface 5 of anchorage surface device 1 may be surrounded by a continuous interstitial surface. By way of example for illustrative purposes, each of the wells of a multi-well plate will include a bottom wall and the entirety of such bottom wall corresponds to a respective one anchorage surface 5 and the interstitial surface corresponds to regions between each anchorage surface 5.

In one embodiment, anchorage surface 5 may comprise a plurality of microspots 50 disposed thereon that may tether biological material(s) and that are surrounded by a continuous interstitial surface 60 that does not tether the biological material(s) of interest (FIG. 4). By way of example for illustrative purposes, each well bottom (i.e. anchorage surface) of a multi-well plate may be micropatterned to include a plurality of microspots surrounded by a continuous interstitial surface.

In some embodiments, bottom wall 30 may include any number of microspots 50a, 50b provided that the number is greater than one. The number of microspots on bottom wall 30 necessarily depends on the size (i.e. area) of each microspot, the area of bottom wall, and the spacing between adjacent microspots. For example, in an embodiment where biological materials are tethered three-dimensional aggregates that grow out of one or more anchorage-dependent cells (as may be present in a cell suspension) or out of a tethered tissue or a tissue fragment, and bottom wall 50 is roughly the area of the bottom wall of a 96-well plate, then bottom wall 50 may include between 10 and 100 microspots. If the biological material(s) are smaller than a three-dimensional aggregate of cells than plenty more microspots may be disposed on bottom wall 50. Likewise, if bottom wall is roughly the area of a 6-well plate and it will be used to grow three-dimensional aggregates (tethered to respective microspots) then plenty more microspots may also be accommodated relative to a 96-well format, for example. Therefore, bottom wall 30 may comprise up to 10 microspots, up to 25 microspots, up to 50 microspots, up to 100 microspots, up to 250 microspots, up to 500 microspots, or up to 1000 microspots, or more.

Microspots 50 can be any shape or size, and their size and shape is desirably tailored to the type of biological material(s) to be tethered thereto. In one embodiment, microspots 50 of an anchorage surface device 1 have the same shape. In one embodiment, microspots 50 are circular. In one embodiment, microspots 50 are elliptical. In one embodiment, microspots 50 are triangular. In one embodiment, microspots 50 are quadrilateral. In one embodiment, microspots 50 may be irregularly shaped, such as in the shape of a tear-drop or a tree/branched structure. In one embodiment, microspots 50 may be shaped to recapitulate a physiological shape, such as in the case of a kidney having tubules or in the case of muscle cells or cardiac muscle cells in parallel linear arrays.

In one embodiment a size of each of the plurality of microspots 50a, 50b, and so on is the same. Depending on the shape of microspots 50, it may be difficult to measure a size thereof. For the purposes of this disclosure references to the size of the microspots generally mean a measurement taken from a first edge of a microspot to the furthest second edge of the same microspot. For example, if the microspot is spherical then the size may be measured across the diameter of the microspot. And, if the microspot is triangular then the size may be measured from a first point to a second point of the microspot.

Each of the plurality of microspots 50a, 50b, and so on are separated by a pitch p from a microspot adjacent thereto. Indeed pitch p of any adjacent two microspots (e.g. 50a and 50b) may be a function of the type of biological material(s) to be tethered thereto. Where the biological material is relatively small, such as a protein or a nucleic acid, a size of each microspot (being the largest measurement taken from a first edge of a microspot to a second edge of the same microspot) may be between about 10 nm to 1 μm, for example. Accordingly, pitch p would be at least about 10 nm to 1μm, and likely greater in order to have some separation between the adjacent two microspots. Where the biological material is relatively larger, such as a tissue fragment or an aggregate of cells, a size of each microspot (being the largest measurement taken from a first edge of a microspot to a second edge of the same microspot) may be between about 20 μm to 2000 μm, for example, and preferably between about 100 μm to 1000 μm. Accordingly, pitch p would be at least about 20 μm to 2000 μm and likely greater in order to have some separation between the adjacent two microspots.

In any case, pitch p may be between about 5 nm to about 10000 μm. In one embodiment pitch p may be between about 10 μm to about 5000 μm. In one embodiment pitch p may be between about 200 μm to about 1000 μm.

In one embodiment, assuming the average diameter of a mammalian cell is approximately 10 μm, and the average diameter of a three-dimensional aggregate of mammalian cells (such as an organoid) is approximately 500 μm, pitch p may be about 750 μm, about 1 mm, about 2 mm, about 3 mm, about 4 mm, or about 5 mm. Thus, pitch p is an important consideration so as to reduce the likelihood that cells (or three-dimensional aggregates) growing on adjacent microspots will fuse, or otherwise come into direct contact with one another.

In addition to pitch p being a function of a respective size of two adjacent microspots, pitch p is also a function of the spacing sp between the two adjacent microspots. Depending on the shape of microspots 50, it may be difficult to measure spacing sp between two adjacent microspots (e.g. 50a and 50b). For the purposes of this disclosure references to the spacing between two adjacent microspots generally means a measurement taken from a first edge of a first microspot to a closest first edge of an adjacent second microspot. For example, if the two adjacent microspots are spherical then the spacing is determined by measuring the distance between the point where the diameter intersects the circumference of a first microspot and the closest point where the diameter intersects the circumference of a second adjacent microspot. And, if for example two adjacent microspots are triangular then the spacing is determined by measuring the distance between a point of the first triangular microspot and the closest point of the second adjacent triangular microspot.

In one embodiment spacing sp between two adjacent microspots taken from a first edge of a first microspot to a closest second edge of an adjacent second microspot is between about 50 μm and 5000 μm. In one embodiment, spacing sp is preferably between about 100 μm and 1000 μm.

Sufficient spacing sp between each of the plurality of microspots 50 is important in order to keep biological material(s) that are tethered to the microspots from directly contacting or fusing with one another. In this regard it is important that the area of bottom wall 30 (or first planar surface 10) that is not covered by the plurality of microspots 50—the continuous interstitial surface 60—does not bind or tether biological material(s). Said differently, each of the plurality of microspots (e.g. 50a, 50b, and so on) is surrounded by continuous interstitial surface 60 that does not support tethering of biological material(s), whereas each of the plurality of microspots (e.g. 50a, 50b, and so on) are capable of supporting tethering of biological material(s).

Plurality of microspots 50 possess a chemical attribute that is different from a chemical attribute of the area making up continuous interstitial surface 60. Thus, the chemical attribute of the plurality of microspots 50 is responsible for the direct or indirect tethering of biological material(s) thereto. In the alternative, the chemical attribute of continuous interstitial surface 60 is responsible for the im permissiveness of tethering biological material(s) thereto.

In one embodiment, the chemical attribute of the plurality of microspots 50 that is different from the chemical attribute of continuous interstitial surface 60 may be characterized as a difference in charge. In the same or different embodiment, the chemical attribute of the plurality of microspots 50 that is different from the chemical attribute of continuous interstitial surface 60 may be characterized as a difference in reactive or functional group. In the same or different embodiment, the chemical attribute of the plurality of microspots 50 that is different from the chemical attribute of continuous interstitial surface 60 may be characterized as a difference in attraction to certain materials, such as aqueous or organic solutions. In the same or different embodiment the chemical attribute of the plurality of microspots 50 that is different from the chemical attribute of continuous interstitial surface 60 may be characterized as a proclivity of microspots 50 to bind a fluid coating supplement and/or protein, thereby supporting the tethering of biological material(s).

Fluid coating supplement may be necessary, depending on the application, to enable tethering of biological material(s) indirectly to the plurality of microspots. For example, if the biological material(s) are tissues, tissue fragments, cells or aggregates of cells then such biological material(s) may require a fluid coating supplement to support their viability (e.g. survival and/or growth and/or development and/or differentiation). For example, many tissue fragments or cells (or aggregates of cells, such as organoids) grow optimally in the presence of a fluid coating supplement such as Matrigel®, laminin(s), vitronectin(s), fibronectin(s), collagen(s), etc. Such fluid coating supplements are widely commercially available. Therefore, in one embodiment the fluid coating supplement is a solution of one or more extracellular matrix proteins. In one embodiment, the fluid coating supplement may include natural extracellular matrix proteins (i.e extracellular matrix proteins produced in, or isolated from, producing cells). In one embodiment, the fluid coating supplement may include synthetic/engineered extracellular matrix protein analogues. For example, synthetic hydrogels and the like are known in the art and are specifically engineered for supporting particular cell types.

Plurality of microspots 50 may be disposed on bottom wall 30 or first planar face 10 using a source of energy, such as ultraviolet light. If using ultraviolet light, plurality of microspots 50 may be formed via a photomask comprising a plurality of apertures therethrough which define the appropriate microspot: size(s); pitch p; spacing sp; number; etc. In one embodiment, the photomask may be made of quartz. Thus, by exposing bottom wall 30 or first planar face 10 to a source of energy, thereby disposing the plurality of microspots 50 thereupon, each of the plurality of microspots 50 embodies a modified chemical attribute relative to continuous interstitial surface 60. In one embodiment, as a result of the modified chemical attribute, a fluid coating supplement is preferentially engaged by the plurality of microspots 50 in comparison to the continuous interstitial surface 60.

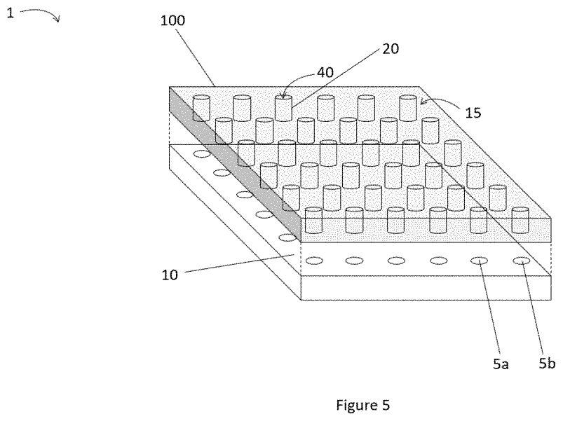

In one embodiment, anchorage surfaces 5 (each of which may further comprise a set of plurality of microspots 50) are formed on first planar face 10 and one or more leak-proof physical barrier 100 is subsequently attached to first planar face 10 (FIG. 5). One or more leak-proof physical barrier 100 circumscribes a respective anchorage surface 5 (or a set of plurality of microspots 50 as may be comprised in the respective anchorage surface 5) to form receptacle 15 when attached to first planar face 10. Thus, one or more leak-proof physical barrier 100 may alternatively be referred to as a receptacle-defining member, which defines one or more side wall 20 and open top end 40 (and bottom wall 30 once attached to first planar face 10).

In one embodiment, one or more leak-proof physical barrier 100 are individually attached to first planar face 10 (to circumscribe a respective anchorage surface 5 or a set of plurality of microspots 50 as may be comprised in the respective anchorage surface 5). In one embodiment, more than one leak-proof physical barrier is comprised in a well-defining member. Thus, each of the more than one leak-proof physical barriers comprised in the well-defining member respectively circumscribe a separate anchorage surface 5 (or separate set of plurality of microspots 50 as may be comprised in the separate anchorage surface 5).

In some embodiments it may be important that anchorage surface device 1, and optionally first planar face 10, possesses sufficient optical clarity to permit imaging of the three-dimensional aggregates formed and anchored thereto.

Below, uses of the anchorage surface devices 1 of the disclosure will be described. Methods and assays

In another aspect of this disclosure are provided methods and/or assays which may be conducted using anchorage surface device 1, as described above.

In one embodiment, are provided methods of tethering biological material(s) to anchorage surfaces 5 or plurality of microspots 50. Such methods may be employed to test growth/tethering conditions or to test different conditions on growing/grown or on tethered biological material(s). In one embodiment, are provided methods/assays using biological material(s) tethered to anchorage surfaces 5 or plurality of microspots 50, and subsequently exposing said biological material(s) to one or more conditions and monitoring the effects of such one or more conditions over time.

One benefit of using the anchorage surface device 1 of this disclosure is the ability to conduct methods/assays while maintaining biological material replicates as distinct and discrete entities in a tethered relationship with respective anchorage surfaces/microspots. In a specific embodiment, the biological material is a three-dimensional aggregate of cells (e.g. organoid), and anchorage surface device 1 enables the tethered three-dimensional growth of such aggregates while being confined to a specific location of anchorage surface device 1. In addition to confining tethered biological material to a specific location of device 1, it is also made possible that tethered biological material may be constrained in terms of overall number (depending on, for example, the number of microspots), size, shape, and location when employing device 1.

Another benefit of using the anchorage surface device 1 of this disclosure in various methods/assays is the ability to conduct such methods/assays on biological materials (that may or may not be three-dimensional) tethered on the same plane, which may facilitate imaging to, for example, track growth/survival of the tethered biological material in response to test and control conditions or to conduct imaging. By tethering the biological material in the same focal plane, progress of growth or response to applied conditions may be accurately and efficiently tracked, such as by imaging that may be automated, over a period of time.

Another benefit of using the anchorage surface devices 1 of this disclosure is the ability to conduct methods/assays while confidently monitoring the effects of one or more conditions on a specific tethered biological material(s) or population of tethered biological material(s), such as over a period of time. Otherwise, it would be impossible, or very difficult, to monitor effects on specific subjects/objects freely dispersed within a liquid or semi-liquid environment. Thus, anchorage surface device 1 may permit a greater resolving power of the effects of a treatment at the level of an individual subject/object, such as over a period of time.

Another benefit of using the anchorage surface devices 1 of this disclosure is the ability to conduct methods/assays on a specific sample size of tethered biological material(s). As indicated, the number of microspots in a well bottom, for example, permit a desired number of unique/discrete data points to be exposed to a common condition (and other well bottoms can be exposed to the same or different common condition). Accordingly, use of device 1 to carry out the methods/assays contemplated herein allows for the acquisition of sufficient biological and technical replicates, so to apply statistical operations on the tested conditions, while nevertheless permitting the effects to be monitored/tracked down to the individual level, such as by imaging over a period of time.

Another benefit of using the anchorage surface devices 1 of this disclosure is the ability for the biological material, and particularly in the case of cells, to survive and grow while tethered to the plurality of microspots 50. Thus, the timing when the assays/methods may be performed is entirely at the control of the user, such as initiating the methods/assays only once the biological material reaches a certain stage of growth or differentiation, or a level of readiness to be experimented upon

Another benefit of using an anchorage surface device 1 of this disclosure is the ability for the biological material, and particularly in the case of cells, to survive and grow while tethered to the plurality of microspots 50 but at the same time restricting its growth into the continuous interstitial space. Thus, biological material associated with one anchorage surface or microspot cannot grow into or fuse with biological material of another anchorage surface or microspot. Further, restricting growth away from or off of the anchorage surface/microspot into the continuous interstitial space appears to reduce the differentiation of off-target cells when, for example, pluripotent stem cells (PSC) are seeded and the PSC are exposed to differentiation conditions, such as to organoid differentiation conditions. By way of concrete example, but not intended to be limiting, constrained tethering of pluripotent stem cells to microspots followed by exposure to conditions for differentiating/forming kidney organoids, less mesoderm and ectoderm arises in comparison to a culture surface that does not include anchorage surfaces/microspots.

Another benefit of using an anchorage surface device 1 of this disclosure is the ability to form the tethered three-dimensional aggregates (e.g. organoids) in the absence of conventional Matrigel (or other fluid coating supplement) sandwich or dome conditions. It is shown herein that rather than sandwiching (or embedding in a dome) seeded cells (or clumps or fragments) between the fluid coating supplement-bound anchorage surface (e.g. microspots) and an upper fluid coating supplement overlay, the cells seeded on the fluid coating supplement-bound anchorage surface (e.g. microspots) may grow into tethered aggregates (e.g. organoids) in the presence of sub-gelation threshold levels of the fluid coating supplement diluted in culture medium, such as between about 0-10%. Minimizing the quantities of fluid coating supplement required helps to reduce experimental/assay costs while also providing the tethered biological materials better access to components in their environment (e.g. nutrients, drugs, compounds, growth factors/cytokines, etc) rather than relying on variable diffusion rates through a gelled fluid coating supplement.

It has previously been described herein that any type of biological material may be tethered to anchorage surfaces 5 or plurality of microspots 50. In one embodiment, the biological material may be a peptide or polypeptide, such as a cellular or synthesized/recombinant protein. In a specific embodiment the protein may be an antibody or an enzyme. Such tethered proteins may be assayed for their ability/inability to bind, cleave, or perform an operation on a component of a test condition relative to a control condition.

In one embodiment, the biological material may be a nucleic acid, such as a cellular or synthesized/recombinant nucleic acid. In a specific embodiment, the nucleic acid may be DNA, RNA, or an oligonucleotide nucleotide. Such tethered nucleic acids may be assayed for their ability/inability to bind, cleave, or perform an operation on a component of a test condition relative to a control condition.

In one embodiment, the biological material may be a tissue or a tissue fragment obtained/derived from a multicellular organism of any species, such as a human or a non-human animal, such as a primate, rodent, or any other type of non-human mammal. In a specific embodiment, the tissue or tissue fragment may be ectodermal tissue, endodermal tissue, embryonic tissue, or mesodermal tissue. Such tethered tissue or tissue fragment may be assayed for responsiveness to a test condition relative to a control condition. Or, such tethered tissue or tissue fragment may be assayed for survival, growth or differentiation in response to a test condition relative to a control condition.

In one embodiment, the biological material may be a tissue or a tissue fragment derived from a healthy, tumorigenic (either primary or metastatic), inflammatory, damaged tissue source or a tissue containing genetic disorders or non-genetic diseased tissue. Such tethered tissue or tissue fragment may be assayed for responsiveness to a component of a test condition relative to a control condition. Or, such tethered tissue or tissue fragment may be assayed for survival, growth or differentiation in response to a test condition relative to a control condition.

In one embodiment, the biological material may be a tissue or a tissue fragment from an adult, or fetal tissue source. Such tethered tissue or tissue fragment may be assayed for responsiveness to a component of a test condition relative to a control condition. Or, such tethered tissue or tissue fragment may be assayed for survival, growth or differentiation in response to a test condition relative to a control condition.

In one embodiment, the biological material may be a cell, a suspension of cells (including a suspension of single cells, a suspension of cell aggregates, a suspension of clumps of cells, or any combination of the foregoing). In one embodiment, the suspension of cells comprises healthy cells, or tumor cells from either primary or metastatic lesions, or cells containing genetic disorders, or cells from inflammatory or non-genetic diseased or damaged tissues, or any combination of the foregoing.

In a specific embodiment, the cell or suspension of cells may be ectodermal, endodermal, embryonic, or mesodermal. In one embodiment, the embryonic cells may by pluripotent stem cells, such as embryonic stem cells or induced pluripotent stem cells. Such tethered cell or one or more cells of the suspension of cells may be assayed for its responsiveness to a test condition relative to a control condition.

Or, such tethered cell or one or more cells of the suspension of cells may be assayed for survival, growth or differentiation in response to a test condition relative to a control condition.

In one embodiment, the biological material is an aggregate of cells. In a specific embodiment, the aggregate of cells is a tethered three-dimensional aggregate of cells. In one embodiment, the aggregate of cells may be ectodermal, endodermal, embryonic, or mesodermal. Such tethered aggregate of cells may be assayed for its responsiveness to a test condition relative to a control condition. Or, such tethered aggregate of cells may be assayed for survival, growth or differentiation in response to a test condition relative to a control condition.

In a specific embodiment, the three-dimensional aggregate of cells is an organoid or a spheroid, which organoid or spheroid is tethered to anchorage surface 5 (or plurality of microspots 50) of anchorage surface device 1 and grows out of one or more anchorage-dependent cells of a cell suspension. Thus, a non-limiting description of methods of growing a three-dimensional aggregate of cells using anchorage surface device 1 is described below.

Typically, adherent (or anchorage-dependent) primary cells, cell lines, pluripotent stem cells (e.g. embryonic stem cells or induced pluripotent stem cells (PSC), or PSC-derived cells rely on a fluid coating supplement applied to the surface upon which they are tasked to grow. The skilled person will appreciate that not all cells will require a coating supplement, however, many cell types such as stem cells, epithelial cells, neural cells, mesenchymal cells or progenitors of any of the foregoing grow and perform more optimally when cultured on a fluid coating supplement. Examples of common fluid coating supplements include laminin(s), proteoglycan(s), fibronectin(s), collagen(s), Matrigel®, etc. Many fluid coating supplements are widely commercially available.

Coating of anchorage surface device 1, or each receptacle 15 thereof, may be carried out by contacting anchorage surface 5 or (each set of) plurality of microspots 50 of first planar face 10 with a fluid coating supplement. Fluid coating supplements are known in the art, and examples are enumerated hereinabove. Depending on the fluid coating supplement, coating usually requires at least a brief incubation period. In any event, contacting anchorage surface 5 or (each set of) plurality of microspots 50 is carried out for a time sufficient to allow some or all of the fluid coating supplement to engage with (i.e. bind) anchorage surface 5 or (each set of) plurality of microspots 50. Again depending on the fluid coating supplement, it may be necessary to remove excess fluid coating supplement after the incubation period.

Given the nature of anchorage surface 5 or (each set of) plurality of microspots 50 relative to continuous interstitial surface 60, fluid coating supplement should preferentially engage (i.e. bind) anchorage surface 5 or (each set of) plurality of microspots 50. Applying a limiting volume of fluid coating supplement may obviate the need to remove/wash excess fluid coating supplement. Nevertheless, if excess fluid coating supplement is removed/washed from device 1 or receptacle 15, the end result should be fluid coating supplement preferentially bound to anchorage surface 5 or (each set of) plurality of microspots 50 and substantially absent from continuous interstitial surface 60 (because fluid coating supplement and/or proteins do not tend to bind to surface 60).

Following the coating step, the first planar face 10 (or well bottom 30) may be contacted with a cell suspension and then culturing the one or more anchorage-dependent cells of the cell suspension in a supportive culture medium under supportive culture conditions.

The cell suspension may be obtained by appropriately processing a tissue sample or an existing culture of cells. In one embodiment the cell suspension is a suspension of PSC, such as a suspension comprising undifferentiated PSC. In one embodiment the cell suspension is a suspension of PSC-derived cells, such as a suspension of differentiated PSC.

In one embodiment the cell suspension is a suspension of primary cells (or tissue-derived cells), such as may be obtained by processing a tissue or a tissue sample. In one embodiment the suspension of primary cells is a dissociated tissue sample or a blood sample. In one embodiment the suspension of primary cells is from an adult, or fetal tissue sample. In one embodiment the dissociated tissue sample is derived from a neural tissue (e.g. a brain tissue, such as a microglial tissue, a choroid plexus tissue, etc.).

In one embodiment the dissociated tissue sample is derived from an epithelial tissue, wherein the epithelial tissue is a lung, a kidney, a pancreas, a liver, a small intestine, a large intestine, a stomach, a prostate, or mammary. In one embodiment the dissociated tissue sample is derived from a tumour sample (or a dissociated culture of cancer cells). In one embodiment the dissociated tissue sample is derived from a primary tumour, or a metastatic lesion. In one embodiment the dissociated tissue sample is derived from an inflammatory, or damaged tissue. In one embodiment the dissociated tissue sample is derived from tissue containing a genetic disorder, or a non-genetic diseased tissue. In one embodiment, dissociated tissue sample includes at least one stem or progenitor cell (i.e. a cell that is capable of giving rise to the tissue).

Culture media that may be used to culture the one or more anchorage-dependent cells of the cell suspension are highly dependent on the type of anchorage-dependent cell to be cultured. The person skilled in the art will appreciate that various manufacturers sell a wide range of cell-type specific media. Alternatively, the person skilled in the art may use a particular culture medium that they formulate on their own. Commercially available culture media for culturing PSC and/or aggregates of PSC include mTeSR™ 1 (STEMCELL Technologies), mTeSR™ 2 (STEMCELL Technologies), TeSR™ E8 (STEMCELL Technologies), mTeSR™ Plus (STEMCELL Technologies), and mTeSR™ 3D (STEMCELL Technologies). Commercially available culture media for growing hepatic organoids are sold under the HepatiCult™ (STEMCELL Technologies) brand. Commercially available culture media for growing intestinal organoids are sold under the IntestiCult™ (STEMCELL Technologies) brand or the STEMdiff™ (STEMCELL Technologies) brand. Commercially available culture medium for growing pancreatic organoids are sold under the PancreaCult™ (STEMCELL Technologies) brand or the STEMdiff™ (STEMCELL Technologies) brand. Commercially available culture media for growing pulmonary organoids are sold under the PneumaCult™ (STEMCELL Technologies) brand or the STEMdiff™ (STEMCELL Technologies) brand. A commercial available culture medium for growing prostate organoids is sold under the ProstaCultTM (STEMCELL Technologies) brand. A commercially available culture medium for growing cerebral organoids is STEMdiff™ Cerebral Organoid Kit (STEMCELL Technologies). A commercially available culture medium for growing chondrocytes from mesenchymal stem cells is MesenCult™-ACF Chondrogenic Differentiation Kit (STEMCELL Technologies). A commercially available culture medium for differentiating and growing PSC-derived intestinal organoids is STEMdiff™ Intestinal Organoid Kit. A commercial available culture medium for differentiating and growing PSC-derived kidney organoids is STEMdiff™ Kidney Organoid Kit (STEMCELL Technologies).

Culture conditions that may be used to culture the one or more anchorage-dependent cells of the cell suspension may also be highly dependent on the type of anchorage-dependent cell to be cultured. While mammalian cells typically grow well at about 37° C. in 5% CO₂, it may be necessary in some circumstances to deviate from these conditions, such as under hypoxia conditions for some tumour cells or cells of tissue exposed to relatively hypoxic conditions. Nevertheless, the duration of cell culture and frequency of medium exchanges may be cell-type specific, in which case it is advisable to at least begin from the manufacturer's recommended practices and if necessary to optimize/troubleshoot from there.

After culturing the one or more anchorage-dependent cells of the cell suspension in contact with the coated first planar face 10, the one or more anchorage-dependent cells may adhere to the coating supplement and begin to grow (in the presence of a suitable culture medium). It bears repeating that meaningful adherence and growth of the one or more anchorage-dependent cells should preferentially or only occur over the anchorage surface 5 or (each set of) plurality of microspots 50 owing to its differential chemical attribute (which may preferentially engage biological materials, such as may be contained in the fluid coating supplement) relative to the chemical attribute of the continuous interstitial surface 60 (which may not preferentially engage biological materials, such as may be contained in the fluid coating supplement).

In some cases it may be necessary to overlay the seeded cells with additional fluid coating supplement. In one embodiment, the fluid coating supplement may be directly applied to the seeded cells before the culture media is added. In one embodiment, the fluid coating supplement may be diluted in culture media, which mixture may then be applied to the seeded cells. In such embodiments the fluid coating supplement may be diluted to between about 0% and 99%. In one embodiment the fluid coating supplement may be diluted to between about 1% and 50%. In one embodiment the fluid coating supplement is diluted to a sub-gelation threshold, such as less than 20%, or preferably between about 1% to 10%.

Whereas utilizing 100% fluid coating supplement overlay down to 5% fluid coating supplement overlay in the methods disclosed herein usually results in the formation of three-dimensional aggregates (organoids), a 0% fluid coating supplement overlay appears in many systems to maintain only a two-dimensional or two dimensional-like morphology. When utilizing a high percentage overlay of fluid coating supplement, such as between 50-100%, naturally the volume selected may render both the anchorage surface 5 or (each set of) plurality of microspots 50 and the continuous interstitial surface 60 covered in gelled fluid coating supplement. However, this is of little consequence because the cells may grow upward into the gelled fluid coating supplement but not into the continuous interstitial surface 60, given the differential chemical attributes of anchorage surface 5 or (each set of) plurality of microspots 50 and surface 60. Further, the shape, pitch, spacing of anchorage surface 5 or (each set of) plurality of microspots 50 also influences the growth of the tethered biological material. Nevertheless, since a high percentage of fluid coating supplement, such as 50-100% of Matrigel, overlay may result in very high background signal when staining and imaging the formed and tethered three-dimensional aggregates, it will usually be beneficial to use a more dilute fluid coating supplement overlay, such as diluting the fluid coating supplement in the culture medium.

Eventually, a three-dimensional aggregate of cells will grow out of one or more anchorage-dependent cells, which three-dimensional aggregate is tethered to a microspot (and the continuous interstitial surface not supportive of anchorage-dependent growth of the one or more anchorage-dependent cells). In one embodiment the three-dimensional aggregate of cells is an embryoid body, such as may have been differentiated from an aggregate of PSC. In one embodiment the three-dimensional aggregate of cells is an aggregate of undifferentiated PSC.

In one embodiment the three-dimensional aggregate of cells is an organoid (e.g. a spheroid). In such an embodiment the organoid may be one of a lung organoid (such as a proximal or distal airway organoid), a kidney organoid, a pancreatic organoid, a liver organoid, a small intestinal organoid (such as a duodenal, an ileal, an jejunal organoid), a large intestinal organoid (such as a cecum, an ascending, a transverse or descending colonic organoid), a stomach organoid (such as a antral or fundal organoid), a prostate organoid, or a mammary organoid.

In some embodiments the organoid may be lumenized. In the same or different embodiments, the organoid may be polarized by virtue of tethering to the anchorage surface device 1 via a single region or site of the organoid.

In some embodiments, the organoids may express markers and perform functions characteristic of the tissue type recapitulated by the organoid. The skilled person in the art will know the type of markers that are characteristic of the particular type. Nevertheless, it is both beneficial and surprising that organoids (or any other type of three-dimensional aggregate of this disclosure) may form while tethered to an underlying surface and also express characteristic markers and perform expected functions. Thus, the tethered three-dimensional aggregates may be used for compound screening, toxicity testing, (forskolin-induced) swelling assays, downstream gene or protein expression assays, etc.

In a specific embodiment, the one or more anchorage-dependent cells are primary kidney stem or progenitor cells or PSC-derived kidney stem or progenitor cells and the three-dimensional aggregates of cells are kidney organoids. In one embodiment the kidney organoid(s) contain fewer neuro-ectodermal cells and stromal cells compared to kidney organoids not grown using anchorage surface device 1. More specifically, kidney organoids formed in a standard 96 well plate exhibit more TUJ1 positive neuro-ectodermal and VIM and M EIS1/2/3 double positive stromal cells throughout the monolayer rather than when kidney organoids are respectively anchored to the microspots, in which case TUJ1 positive and VIM and M EIS1/2/3 double positive cells, if any, are restricted to the organoids.

In one embodiment the three-dimensional aggregate of cells is a mass of chondrocytes. In one embodiment, the one or more anchorage dependent cells are mesenchymal stem cells or PSC-derived mesenchymal stem cells and the three-dimensional aggregate is a mass of chondrocytes.

Therefore, a benefit of growing a three-dimensional aggregate of cells in accordance with the methods described herein is that the tethered three-dimensional aggregate contains reduced off-target cell differentiation compared to three-dimensional aggregates not grown using anchorage surface device 1. As described hereinabove, off-target cell differentiation refers to cell types (or the quantity of such cell types) arising during culture that are not the expected or are undesired when culturing under the particular conditions used.

An additional benefit of growing tethered three-dimensional aggregates of cells in accordance with methods described herein is that they are formed with a consistent size, shape, and position in comparison to three-dimensional aggregates formed using a standard (non-microspotted) cell culture device. Such factors enable automated detection of the three dimensional aggregates (e.g. organoids) via image analysis, which allows for quantification of marker expression within each organoid, automated counting of aggregate formation efficiency, and determination of their surface area, volume, and/or diameter.

A still further benefit of growing tethered three-dimensional aggregates of cells in accordance with methods described herein is that such three-dimensional aggregates of cells may be better suited for applications where it is preferable to deplete or minimize the numbers of off-target cells prior to performing downstream assays, such as single cell sequencing of target cell types (e.g exclusively PSC-derived organoids of interest, such as kidney organoids) or drug screening (e.g. of target, such as PSC-derived organoids).

Regardless of the type of three-dimensional aggregate of cells grown using devices and methods as disclosed herein, if properly maintained each tethered three-dimensional aggregate of cells may continue to grow until it becomes necrotic or fuses with an adjacent three-dimensional aggregate of cells, or it is utilized in downstream assays. However, it is generally not advisable to grow the tethered three-dimensional aggregates of cells to the point of necrosis, fusion, or the like.

In one embodiment, downstream applications may comprise exposing each anchorage surface 5 or receptacle 15 and the tethered biological material(s) (or three-dimensional aggregate(s) of cells) to a test condition. Similarly, a downstream assay may encompass exposing a first biological material (or three-dimensional aggregate of cells) to a first condition and exposing a second biological material (or three-dimensional aggregate of cells) to a second condition. In one embodiment the first condition is a test condition and the second condition is a control condition. The skilled person will appreciate that the only limit upon the number of conditions to assay is the number of anchorage surfaces 5 or receptacles 15, whether of one or more anchorage surface devices 1. In one embodiment, the first biological material is the same as the second biological material, and in one embodiment they are different. In one embodiment, the first condition is the same as the second condition, and in one embodiment they are different.

After exposing the tethered three-dimensional aggregates of cells to one or more conditions, the method/assay may further comprise monitoring effects of the first condition on the first three-dimensional aggregate and a second condition on the second three-dimensional aggregate. In one embodiment, the first condition and the second condition (or the one or more conditions) are monitored over a period of time.

In one embodiment, a test condition (i.e. the first condition) encompasses, for example, growing the tethered three-dimensional aggregate of cells in the presence of a modified medium formulation or a particular drug or compound. In such an embodiment, the control condition (i.e. the second condition) encompasses, for example, growing the three-dimensional aggregate of cells in the presence of the original (unmodified) medium formulation or in the absence of the particular drug or compound.

In one embodiment, a test condition (i.e. the first condition) encompasses, for example, exposing a formed and tethered three-dimensional aggregate of cells to a differentiating/activating medium formulation or a particular drug or compound. In such an embodiment, the control condition (i.e. the second condition) encompasses, for example, growing the three-dimensional aggregate of cells in the presence of the original (growth) medium formulation or in the absence of the particular drug or compound.

Accordingly, the present disclosure provides devices for tethering biological material(s) thereto, methods of tethering biological materials (e.g. growing a three-dimensional aggregate of cells) to the devices of the disclosure, and assays using the biological materials (e.g. three-dimensional aggregates of cells) tethered to the devices of the disclosure.

As described above, anchorage surface device 1 may enable various applications/assays using one or more test conditions and control conditions where biological and technical replicates are desired in order to assess statistical significance.

Further, anchorage surface device 1 facilitates (as in the methods disclosed herein) limiting an amount of otherwise costly materials, such fluid coating supplement. In one example, Matrigel or other specialized fluid coating supplements are expensive reagents and the methods disclosed herein may reduce the quantities needed by obviating dome or sandwiched culture conditions. Furthermore, including the fluid coating supplement at a sub-gelation threshold in culture medium or any other liquid for bathing biological material, for example, may permit tethered biological to better access nutrients in medium or treatment conditions (be they growth factors/cytokines, drugs/compounds, reagents for staining, etc) and may also minimize corresponding edge effects (e.g. comparing cells at edges vs centre). Also, less cultureware may be used to achieve the same number of data points in an assay because the number of biological materials (e.g. tethered three-dimensional aggregates) assayed directly correlates with the number of anchorage surfaces (i.e. microspots) disposed per receptacle.

Further, anchorage surface device 1 facilitates downstream method steps where culture medium changes and/or supplementation would otherwise cause the loss/displacement of biological materials. A particular benefit of the methods disclosed herein arises in low or high throughput applications, such as drug screens or toxicity testing, where the biological materials are desirably imaged (in the same focal place) throughout or at the end of the methods.

Importantly, anchorage surface device 1 disclosed herein supports the growth or assaying of biological materials only as tethered to a specific anchorage surface 5 or microspot with no tethering to the continuous interstitial surface 60. In this way, aggregates are formed at distinct locations and their growth or health can be tracked during the course of experimentation.

The following non-limiting examples are illustrative of the present disclosure.

### EXAMPLES

### Example 1

### Activating and Coating the Anchorage Surface Device

The anchorage surface device is first manufactured or sourced from a commercial supplier (such as TissueX Technologies or STEMCELL Technologies).

Where the device is commercially sourced, they may be pre-activated. Otherwise, the anchorage surface device may be activated using a solution containing 1-Ethyl-3-[3-dimethylaminopropyl]carbodimide hydrochloride (EDC) and N-hydroxysuccinimide (NHS) in water applied to each receptacle (i.e. well) thereof. After 30 min incubation at room temperature, the wells are washed at least twice with water. After activating the device, a coating solution of about 1% diluted Matrigel® (Corning) is then added to the wells, and the plate is incubated with this solution for at least 6h at 4° C. After this incubation step, the wells are washed at least 3 times with PBS. At this point, the plate has the coating of choice patterned onto the plurality of microspots (but not the continuous interstitial surface), and is ready to be seeded with the biological material of choice.

### Example 2

### Seeding Cancer Cells onto a Coated Anchorage Surface Device

To seed cells on the coated anchorage surface device, a single cell or tissue fragment suspension is first generated and then added to the surface. Note a single cell suspension is preferred. If the receptacle comprising the microspots contains PBS, the PBS should be removed prior to adding the single or tissue fragment suspension.

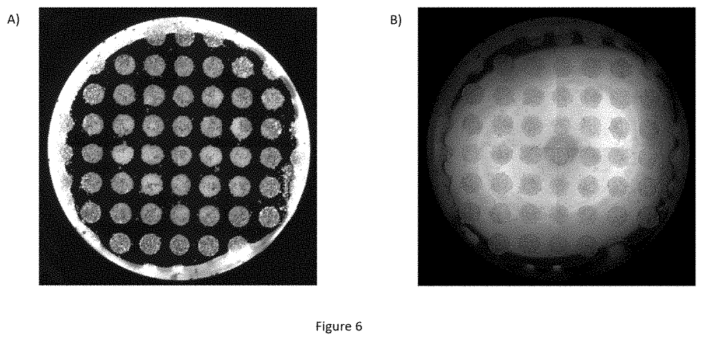

As an example, the anchorage surface device can be seeded with a suspension of single cells of the A549 (ATCC® CCL185™) lung carcinoma cell line and cultured essentially as recommended by the provider. FIG. 6A shows an image of day 2 A549 cells stained with Hoechst and imaged using a fluorescent microscope at 2× magnification. FIG. 6B shows a phase contrast image corresponding with FIG. 6A.

To prepare the suspension of cells, the culture medium is aspirated from the cells and the cells are washed with PBS. After removing the PBS, a trypsin solution (or an alternative single cell dissociation reagent such as Accutase®) is added to the culture and the culture is incubated until the cells appear dissociated, preferably at 37° C. For most trypsin-containing solutions 5-10 minutes is usually sufficient. Once the cells have dissociated, the single cell dissociation reagent is quenched using an appropriate volume of a serum-containing medium. The dissociated cells are triturated several times to generate a single cell suspension, pelleted down, and then resuspended to approximately 1000 cells per 1 μl in cancer cell line maintenance media.

Such a cell suspension is then transferred onto the coated anchorage surface device (i.e. into one or more receptacles thereof) and returned to the 37° C. incubator. For a 96-well plate, 100 μl of the cell suspension can be added to each well. After the cells have settled, typically after 24 h, the media may be replaced with fresh maintenance media and returned to the 37° C. incubator until such time when the cells have achieved a confluence (on the microspots) suitable for downstream applications.

### Example 3

### Seeding hPSCs onto a Coated Anchorage Surface Device

As an additional example, the anchorage surface device can be seeded with a single cell suspension (or clump suspension) of human PSCs. To prepare the cell suspension, the culture medium is aspirated from a maintained culture of the cells and the cells are washed with PBS. After removing the PBS, a trypsin solution (or an alternative single cell dissociation reagent such as Accutase®) is added to the culture and the culture is incubated until the cells appear dissociated, preferably at 37° C. For most trypsin-containing solutions 5-10 minutes is usually sufficient. Once the cells have dissociated, the single cell dissociation reagent may be quenched using an appropriate volume of a hPSC maintenance medium, for example with mTeSR 1™ (STEMCELL Technologies). Note for most hPSCs a ROCK inhibitor such as Y-27632 is recommended to limit cell death while in a single cell suspension. The dissociated cells are triturated several times to generate a substantially single cell (or clump) suspension, pelleted down, and then resuspended to approximately 1000 cells per 1 μl in maintenance media with ROCK inhibitor, for example mTeSR 1™ (STEMCELL Technologies) with 10 μM Y-27632 (STEMCELL Technologies).

Such a cell suspension is then transferred onto the coated anchorage surface device (i.e. into one or more receptacles thereof) and returned to the 37° C. incubator. For a 96-well plate, 100 μl of the cell suspension can be added to each well, but at a sufficient density such that after approximately 24hours in culture one adhered cell should be in contact with at least one other adhered cell. After the approximately 24 hour period of time, the media may be replaced with fresh maintenance media without ROCK inhibitor and returned to the 37° C. incubator until such time when the cells have achieved a confluence (on the microspots) suitable for downstream applications.

### Example 4

### Growing Three-Dimensional Kidney Organoids from PSC on a Coated Anchorage Surface Device

After coating and activating an anchorage surface device in accordance with Example 1, a differentiation protocol can be followed to generate patterned, differentiated cells, or three-dimensional aggregates of differentiated cells tethered to the anchorage surface (e.g. to the microspots). For example, the STEMdiff™ Kidney Organoid Kit (STEMCELL Technologies) can be used to generate three-dimensional kidney organoids on a Matrigel coated anchorage surface device.

Human PSCs previously maintained on mTeSR™ 1 were seeded onto a standard 96-well plate coated with Matrigel and also a micropatterned (i.e. microspotted) 96-well plate coated with Matrigel, as described in Example 3. After 24 hours, adherent cells were overlaid with an additional layer of Matrigel (which eventually resulted in the formation of cavitated PSC spheroids within 48 hours). Differentiation was initiated by switching the medium from mTeSR™ 1 to STEMdiff™ Kidney Organoid Kit media (STEMCELL Technologies). During the next 18 days of differentiation the cells were directed through stages of late primitive streak, posterior intermediate mesoderm, and metanephric mesoderm to give rise to kidney organoids composed of endothelial cells, podocytes, and proximal and distal tubules. (gene expression data not shown)

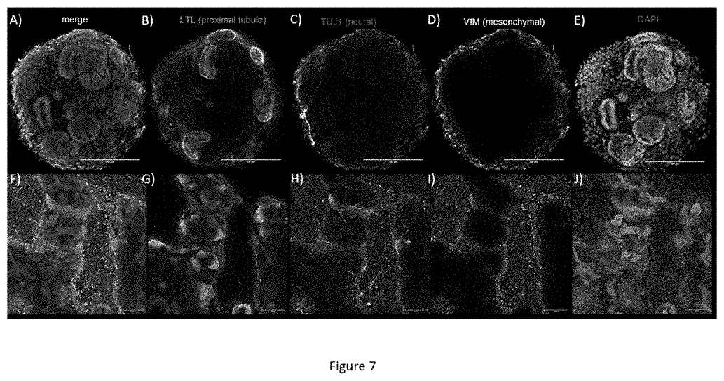

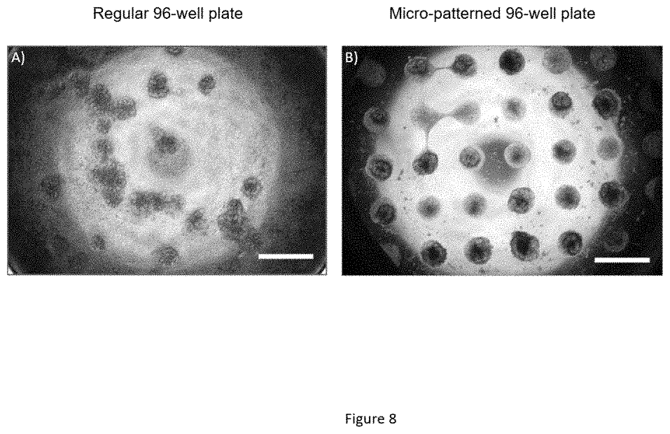

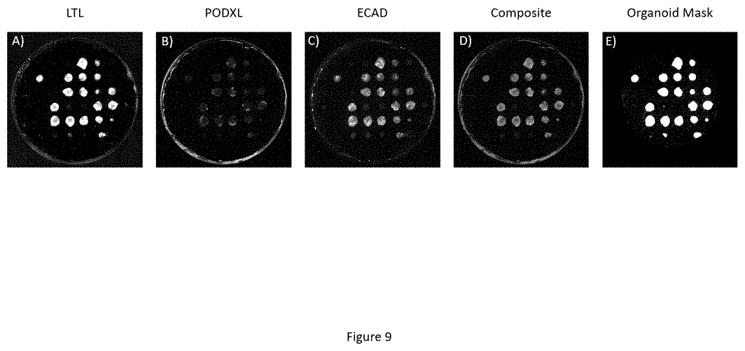

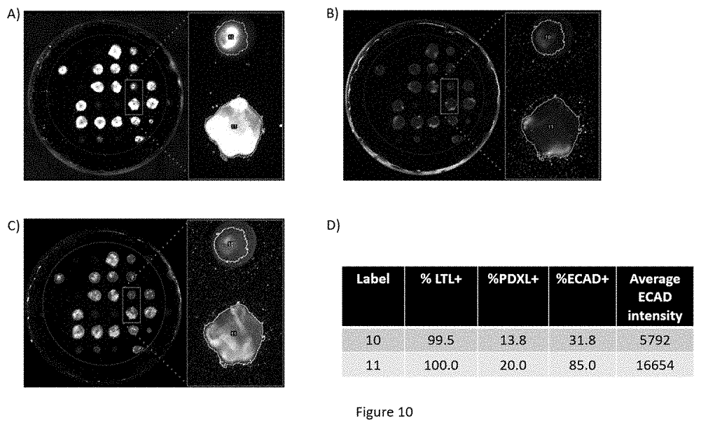

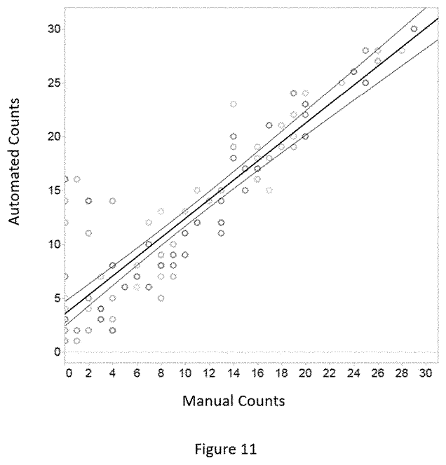

Importantly, when kidney organoid differentiation is performed on patterned 96-well plates there is a marked reduction in off-target cells such as TUJ1-expressing neuro-ectodermal cells and VIM and MEIS1/2/3 double positive mesenchymal cells (FIG. 7A-E, or not shown) in comparison to kidney organoids differentiated in standard 96-well plates (FIG. 7F-J, or not shown). Additionally, the patterned cultures result in kidney organoids of much more consistent size, shape, and position (FIG. 8). Such factors enable automated detection of kidney organoids via image analysis, which allows for quantification of marker expression within each organoid (FIG. 9, FIG. 10). Image-based automated cell counts are comparable to manual counts but can be performed in a fraction of the time (FIG. 11).

### Example 5

### Seeding Adult Stem or Progenitor Cells onto a Coated Anchorage Surface Device

As an additional example, the anchorage surface device (coated and activated in accordance with Example 1) can be seeded with a single cell or cell fragment suspension of adult stem or progenitor cell-containing tissue samples (i.e. tissue-derived cells). Specific examples of adult stem or progenitor cell cultures that can be patterned on the coated anchorage surface device include tissue-derived mouse liver progenitor cells, tissue-derived human and mouse small intestinal cells, tissue-derived human colonic cells, tissue-derived human small intestinal cells, tissue-derived adult mouse prostate cells, and tissue-derived pulmonary (airway) cells.

To prepare the suspension of adult stem or progenitor cells, culture medium is aspirated from a culture of the adult stem or progenitor cells and the cells are washed with PBS. After removing the PB, a trypsin solution (or an alternative single cell dissociation reagent such as Accutase® or TrypLE or Gentle Cell Dissociation Medium) supplemented with or without DNAse is added to the culture, the culture is triturated and is incubated until the adult stem or progenitor cell-containing tissues or aggregates appear dissociated, preferably at 37° C. and with gentle rocking. For most trypsin-containing solutions 5-10 minutes is usually sufficient. Once the cells have dissociated, the single cell dissociation reagent is quenched using an appropriate volume of a serum-containing media, as exemplified in the Examples that follow. Examples of appropriate media product brands include: IntestiCult™, PancreaCult™, HepatiCult™, ProstaCult™, PneumaCultT™, STEMdiff™, etc, each commercially available from STEMCELL Technologies.

The dissociated cells are triturated several times to generate a single cell suspension or a cell fragment suspension, pelleted down, and then resuspended to approximately 1000 cells per 1 μl in adult stem cell culture growth medium.

Such a cell or fragment suspension is then transferred onto the coated anchorage surface device (e.g. into one or more receptacles thereof) and returned to the 37° C. incubator. For seeding single cells, it may be beneficial to include approximately 10 u.M Y-27632 in culture medium used to seed the cells. For a 96-well plate, 100 μl of the cell suspension can be added to each well. After the cells have settled, typically after 24 h, the media may be replaced with specialized adult stem/progenitor cell culture growth media (without ROCK inhibitor, if it was used during initial stages) and returned to the 37° C. incubator until such time when the cells have achieved a state and/or confluence (on the microspots) suitable for downstream applications.

### Example 6

### Growing Three-Dimensional Aggregates from Adult Stem or Progenitor Cells on a Coated Anchorage Surface Device

A single cell suspension or suspension of cell fragments of adult stem or progenitor cells may be patterned on a coated anchorage surface device (coated and activated in accordance with Example 1) as described in Example 5, and such patterned biological material may be subjected to a protocol for generating tethered two-dimensional patterned adult stem or progenitor cells, or tethered three-dimensional aggregates (or organoids) of adult stem or progenitor cells.

After culturing the adult stem or progenitor cells for approximately 24-48 hours, fresh adult stem/progenitor cell culture growth media was added (see the Examples that follow for more specific details about culture conditions). The formation of three-dimensional structures may be promoted by optionally including between 0 and 100%, and preferably 1-5%, Matrigel diluted in appropriate culture medium. Typically, three-dimensional aggregates (or organoids) of adult stem or progenitor cells will begin to appear after about 2 days in culture. The plate set-up including seeded cell number and % Matrigel overlay (MG) is summarized in the table below.

Tissue
type

| | | | | | | | | | | | | |
|---|---|---|---|---|---|---|---|---|---|---|---|---|
| Mouse | 5K | 25K | 50K | 5K | 25K | 50K | 2.5K | 10K | 25K | 2.5K | 10K | 25K |
| Liver | cells | cells | cells | cells | cells | cells | frags | frags | frags | frags | frags | frags |
| | 100% | 100% | 100% | 5% | 5% | 5% | 100% | 100% | 100% | 5% | 5% | v |
| | MG | MG | MG | MG | MG | MG | MG | MG | MG | MG | MG | |
| Human | 5K | 25K | 50K | 5K | 25K | 50K | 200 | 500 | 1K | 200 | 500 | 1K |
| Colon | cells | cells | cells | cells | cells | cells | frags | frags | frags | frags | frags | frags |
| | 100% | 100% | 100% | 5% | 5% | 5% | 100% | 100% | 100% | 5% | 5% | 5% |
| | MG | MG | MG | MG | MG | MG | MG | MG | MG | MG | MG | MG |
| Human | 5K | 25K | 50K | 5K | 25K | 50K | 200 | 500 | 1K | 200 | 500 | 1K |
| Ileum | cells | cells | cells | cells | cells | cells | frags | frags | frags | frags | frags | frags |
| | 100% | 100% | 100% | 5% | 5% | 5% | 100% | 100% | 100% | 5% | 5% | 5% |
| | MG | MG | MG | MG | MG | MG | MG | MG | MG | MG | MG | MG |
| Mouse | 5K | 15K | 25K | 35K | 45K | 55K | 5K | 15K | 25K | 35K | 45K | 55K |
| Prostate | cells | cells | cells | cells | cells | cells | cells | cells | cells | cells | cells | cells |
| | 100% | 100% | 100% | 100% | 100% | 100% | 5% | 5% | 5% | 5% | 5% | 5% |
| | MG | MG | MG | MG | MG | MG | MG | MG | MG | MG | MG | MG |
| Human | 2K | 10K | 20K | 40K | 2K | 10K | 20K | 40K | 2K | 10K | 20K | 40K |
| Airway | cells | cells | cells | cells | cells | cells | cells | cells | cells | cells | cells | cells |
| | 100% | 100% | 100% | 100% | 5% | 5% | 5% | 5% | 0% | 0% | 0% | 0% |
| | MG | MG | MG | MG | MG | MG | MG | MG | MG | MG | MG | MG |

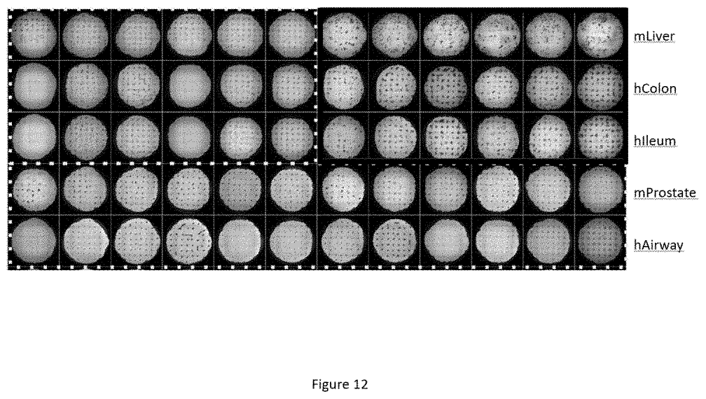

The foregoing procedures were employed to pattern organoids from tissue-derived mouse liver progenitor cells, tissue-derived human colonic cells, tissue-derived human small intestinal cells, tissue-derived airway cells and tissue-derived adult mouse prostate cells (FIG. 12). Additionally, pancreatic progenitor cells derived from PSC can also form into tethered, patterned aggregates (organoids) (Example 13). In the example shown the organoid cultures were grown on the same multi-well culture vessel enabling, among other things, drug screening and toxicity assays to be performed across multiple organoids in a consistent and high-throughput manner.

### Example 7

### Live-Cell Tracking Adult Stem or Progenitor Cell-Derived Organoids on Coated Anchorage Surface Device

The optical clarity of the anchorage surface device coupled with the spatial isolation of aggregates that have been formed thereupon (such as in accordance with Example 6) renders the coated anchorage surface device amenable to live-cell imaging and tracking individual aggregates over time.

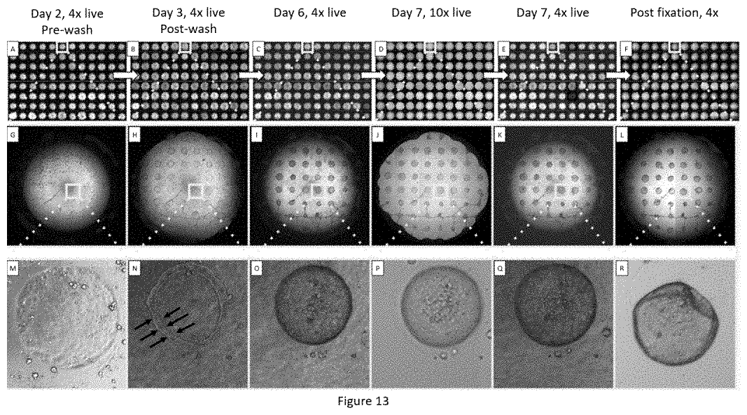

A time course of the culture using the anchorage surface device of FIG. 12 is shown in FIG. 13 A-F. Tissue-derived mouse liver progenitor cells, tissue-derived human colonic cells, tissue-derived human small intestinal cells, tissue-derived adult mouse prostate cells, and tissue-derived human pulmonary cells were used to generate aggregates (organoids) as described in Example 6, and live-cell imaged at multiple time-points: Day 2 (FIG. 13 A, G, M), Day 3 (FIG. 13 B, H, N), Day 6 (FIG. 13 C, I, 0), Day 7 (FIG. 13 D, E, J, K, P, Q). The culture was also imaged post-fixation (FIG. 13 F, L, R).

Whole-well images may also be obtained and singled out for further purpose. For example, FIGS. 13 G-L show zoomed in images of the same receptacle (i.e. well) of the anchorage surface device over the time course. At a higher resolution, FIG. 13 M-R show an individual microspot of the plurality of microspots within a receptacle and the formation of an individual liver aggregate during the time course: after seeding (FIG. 13M); after the wash step where lumen generation may be observed (FIG. 13N); where a hollow sphere is observed (FIG. 13D, P, Q); and post-fixation where the liver aggregate (or organoid) hollow spheres can be seen to collapse (FIG. 13R).

### Example 8

### Growing Three-Dimensional Liver Organoids from Adult Stem or Progenitor Cells on a Coated Anchorage Surface Device

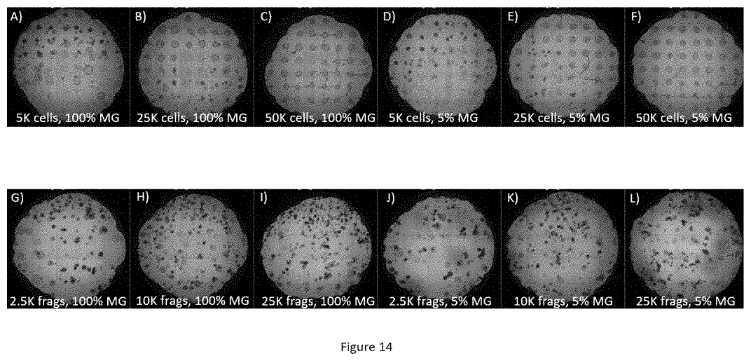

Mouse liver progenitor cells were seeded and grown for 7 days on the plurality of microspots disposed on the anchorage surface device (i.e. a 96-well patterned plate) in accordance with Examples 5 and 6 (FIG. 14). In this embodiment, each microspot had a diameter of about 500 um and arrayed with a pitch of about 700 μm.

Liver progenitor cells were seeded as single cells (FIG. 14A-F) in varying numbers: 5000 cells (A and D); 25000 cells (B and E); and 50000 cells (C and F). Liver fragments were seeded in varying numbers: 2500 fragments (FIG. 14 G and J); 10000 fragments (FIGS. 14 H and K); and 25000 fragments (FIG. 14 I and L). The cells/fragments were cultured in HepatiCult™ Organoid Growth Medium (Mouse). Additionally, the effect of 100% (FIG. 14A-C and G-I) Matrigel overlay and 5% (FIG. 14 D-F and K-L) Matrigel overlay diluted in HepatiCult™ Organoid Growth Medium (Mouse) was also tested.

Subsequent experiments also showed that human liver progenitor cells could be seeded in an anchorage surface device and cultured (essentially as described above) in HepatiCult™ Organoid Growth Medium (Human) (STEMCELL Technologies); if desired, after 2-5 days the tethered aggregates (organoids) can be further matured in HepatiCult™ Organoid Differentiation Medium (STEMCELL Technologies).

We observed single cells seeded (adhered) much better than seeding (adhering) cell fragments. Also, seeding approximately 50000 cells per well resulted in better patterning in comparison to seeding 5000 cells; if fewer than 50000 cells are seeded they can be cultured for a time sufficient so that desired densities are achieved on the microspot(s) before switching to Matrigel-containing media. Additionally, there was no immediately apparent effect on morphology from overlaying the seeded cells with either 5% or 100% Matrigel overlays. Subsequent experiments established that as low as about 2% Matrigel diluted in culture medium could also yield tethered organoids. However, a 100% Matrigel overlay may tend to obscure imaging the cultures (whether phase contrast or confocal).

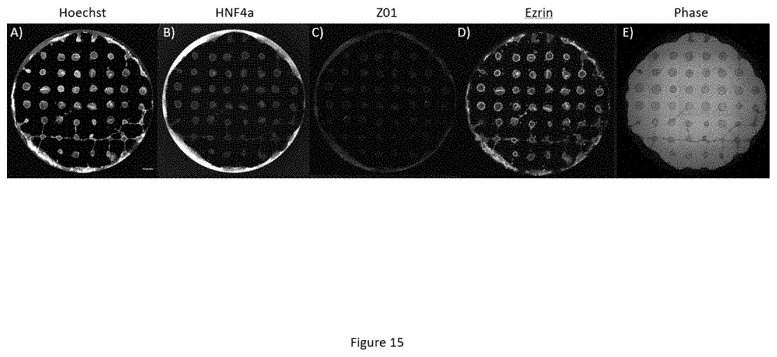

The liver aggregates were further characterized by staining for the liver-specific transcription factor Hepatocyte nuclear factor 4 alpha (HNF4a), the tight junction protein Zonula occludens-1 (Z01), and the membrane cytoskeleton crosslinking protein Ezrin which is expressed apically in liver organoids (FIG. 15).

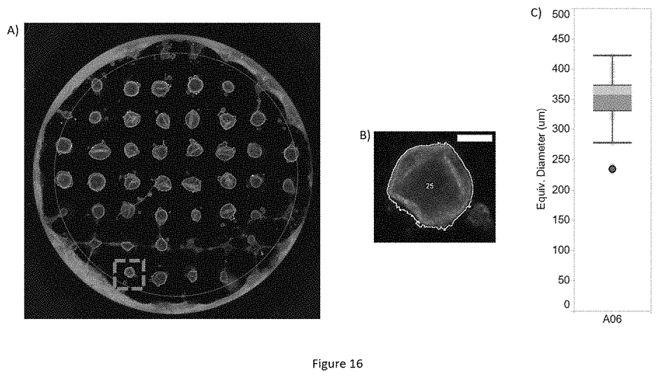

Taking advantage of the spatial segregation of the aggregates, an image analysis algorithm was developed to automatically count and assess the formed aggregates. FIG. 16 shows one receptacle (i.e. well) and the liver aggregates anchored to respective microspots thereof. The aggregates were stained with the DNA fluorochrome Hoechst 33258 and antibodies against HNF4a, Z01, and Ezrin. The superposition of these markers was used to detect the aggregates which could then be assessed for characteristics such as area (expressed in equivalent diameter) and marker expression. The particular aggregate highlighted in the dashed box exhibits an area of about 43012 um², which corresponds to an equivalent diameter of about 234 um which appeared to be an outlier within the distribution of equivalent diameters across all formed liver aggregates.

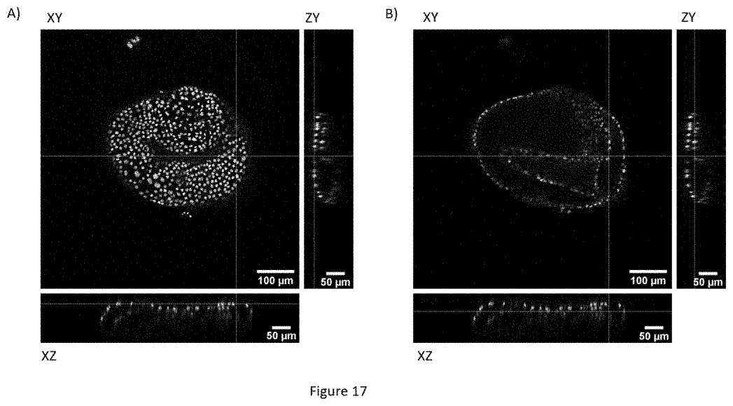

In the time course experiment set out in FIG. 12 and FIG. 13, we observed that seeded liver progenitor cells spontaneously form hollow spheres, with a thickness of one cell layer. To further characterize the aggregates formed on the plurality of microspots, they were scanned using a confocal microscope. Orthogonal sections taken of an exemplary day 7 liver aggregate formed from 50000 cells seeded in a well of a 96-well patterned anchorage surface device and cultured with 5% Matrigel diluted in culture medium that was stained with the DNA fluorochrome Hoechst 33258 clearly show the 3D structure (FIG. 17).

### Example 9

### Growing Three-Dimensional (Large) Intestinal Organoids from Adult Stem or Progenitor Cells on a Coated Anchorage Surface Device

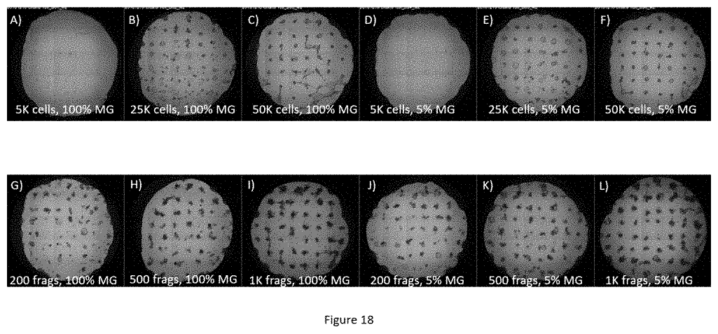

Tissue-derived human (large) intestinal cells were seeded and grown for 7 days on the plurality of microspots disposed on the anchorage surface device (i.e. a 96-well patterned plate) in accordance with Examples 5 and 6 (FIG. 18). In this embodiment, each microspot had a diameter of about 500 um and arrayed with a pitch of about 700 um.

Tissue-derived human intestinal cells were seeded as single cells (FIG. 18A-F) in IntestiCult Organoid Medium (STEMCELL Technologies) varying numbers: 5000 cells (A and D); 25000 cells (B and E); and 50000 cells (C and F). Tissue-derived human intestinal fragments were seeded in varying numbers: 200 fragments (FIG. 18 G and J); 500 fragments (FIGS. 18 H and K); and 1000 fragments (FIG. 18 I and L). Additionally, the effect of 100% (FIG. 18A-C and G-I) Matrigel overlay and 5% (FIG. 18 D-F and K-L) Matrigel overlay diluted in IntestiCult Organoid Medium (STEMCELL Technologies) was also tested.

We observed single cells seeded much better than seeding cell fragments. Also, seeding approximately 50000 cells per well resulted in better patterning in comparison to seeding 5000 or 25000 cells; if fewer than 50000 cells are seeded they can be cultured for a time sufficient so that desired densities are achieved on the microspot(s) before switching to Matrigel-containing media. Additionally, there was no immediately apparent effect on morphology from overlaying the seeded cells with either 5% or 100% Matrigel overlays. However, a 100% Matrigel overlay may tend to obscure imaging the cultures (whether phase contrast or confocal).

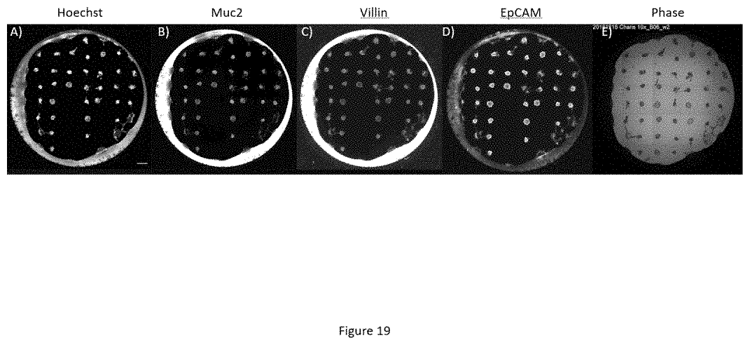

The tissue-derived human colonic aggregates were further characterized by staining with the DNA fluorochrome Hoechst 33258 and antibodies against Muc2, Villin, and EpCAM to detect the presence of goblet cells, enterocytes and intestinal epithelial cells, respectively (FIG. 19).

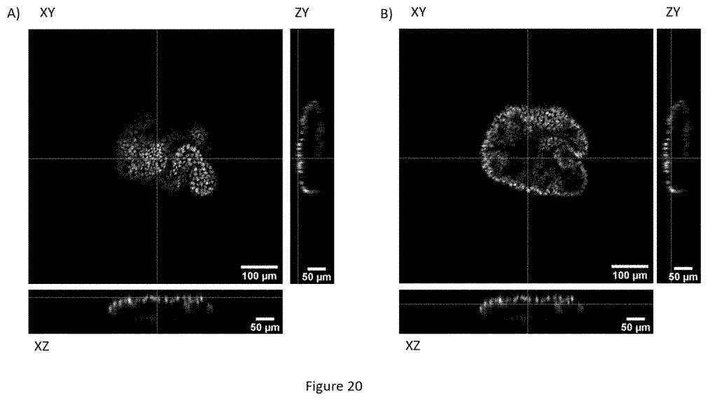

In the time course experiment set out in FIG. 12 and FIG. 13, we observed that seeded tissue-derived human colonic cells spontaneously formed hollow spheres, with a thickness of one cell layer. To further characterize the aggregates formed on the plurality of microspots, they were scanned using a confocal microscope. Orthogonal sections taken of an exemplary day 7 colonic aggregate formed from 50000 cells seeded in a well of a 96-well patterned anchorage surface device and cultured with 5% Matrigel diluted in culture medium that was stained with the DNA fluorochrome Hoechst 33258 clearly show the 3D structure (FIG. 20).

### Example 10

### Growing Three-Dimensional Tissue-Derived Human Small Intestinal Organoids on a Coated Anchorage Surface Device

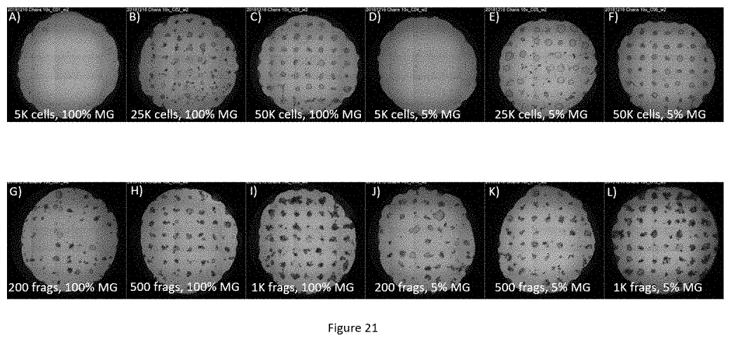

Tissue-derived human small intestinal cells were seeded and grown for 7 days on the plurality of microspots disposed on the anchorage surface device (i.e. a 96-well patterned plate) in accordance with Examples 5 and 6 (FIG. 21). In this embodiment, each microspot had a diameter of about 500 um and arrayed with a pitch of about 700 um.

Tissue-derived human small intestinal cells were seeded as single cells (FIG. 21A-F) in IntestiCult™ OGM Human (STEMCELL Technologies) in varying numbers: 5000 cells (A and D); 25000 cells (B and E); and 50000 cells (C and F). Tissue-derived human small intestinal fragments were seeded in varying numbers: 200 fragments (FIG. 21 G and J); 500 fragments (FIGS. 21 H and K); and 1000 fragments (FIG. 21 I and L). Additionally, the effect of 100% (FIG. 21A-C and G-1) Matrigel overlay and 5% (FIG. 21 D-F and K-L) Matrigel overlay diluted in IntestiCult™ OGM Human was also tested.

We observed single cells seeded much better than seeding cell fragments. Also, seeding approximately 50000 cells per well resulted in better patterning in comparison to seeding 5000 or 25000 cells; if fewer than 50000 cells are seeded they can be cultured for a time sufficient so that desired densities are achieved on the microspot(s) before switching to Matrigel-containing media. Additionally, there was no immediately apparent effect on morphology from overlaying the seeded cells with either 5% or 100% Matrigel overlays. However, a 100% Matrigel overlay may tend to obscure imaging the cultures (whether phase contrast or confocal).

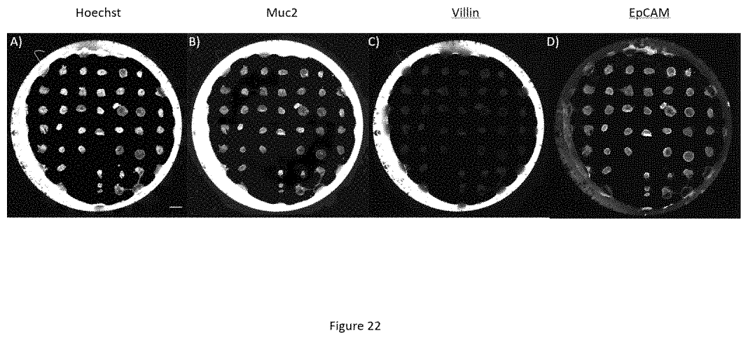

The tissue-derived human small intestinal aggregates were further characterized by staining with the DNA fluorochrome Hoechst 33258 and antibodies against Muc2, Villin, and EpCAM to detect the presence of goblet cells, enterocytes and intestinal epithelial cells, respectively (FIG. 22).

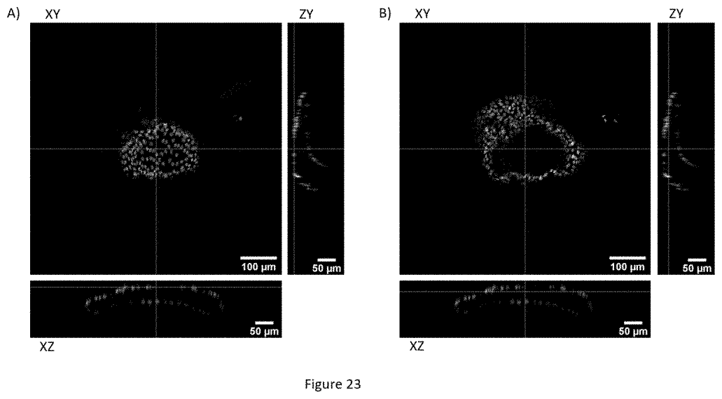

In the time course experiment set out in FIG. 12 and FIG. 13, we observed that seeded tissue-derived human small intestinal cells spontaneously formed hollow spheres, with a thickness of one cell layer. To further characterize the aggregates formed on the plurality of microspots, they were scanned using a confocal microscope. Orthogonal sections taken of an exemplary day 7 small intestinal aggregate formed from 50000 cells seeded in a well of a 96-well patterned anchorage surface device and cultured with 5% Matrigel diluted in culture medium that was stained with the DNA fluorochrome Hoechst 33258, clearly show the 3D structure (FIG. 23).

### Example 11

### Growing Three-Dimensional Tissue-Derived Adult Mouse Prostate Organoids on a Coated Anchorage Surface Device

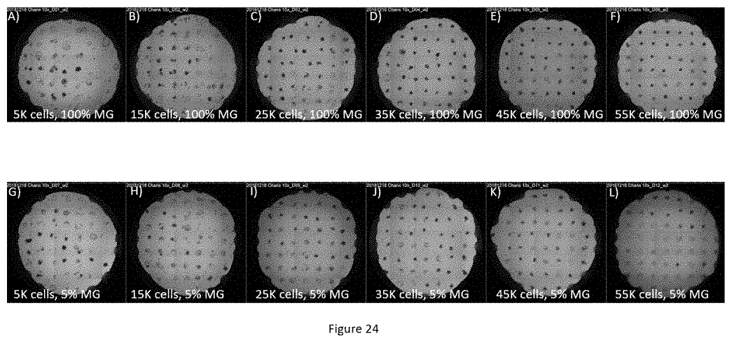

Tissue-derived adult mouse prostate cells were seeded and grown for 7 days on the plurality of microspots disposed on the anchorage surface device (i.e. a 96-well patterned plate) in accordance with Examples 5 and 6 (FIG. 24). In this embodiment, each microspot had a diameter of about 500 um and arrayed with a pitch of about 700 um.

The adult mouse prostate cells were seeded as single cells (FIG. 24A-L) in ProstaCult™ Organoid Medium (STEMCELL Technologies) in varying numbers: 5000 cells (A and G); 15000 cells (B and H); 25000 cells (C and I); 35000 cells (D and J); 45000 cells (E and K); and 55000 cells (F and L). Additionally, the effect of 100% (FIG. 24A-F) Matrigel overlay and 5% (FIG. 24 G-L) Matrigel overlay diluted in ProstaCult™ medium was also tested.

We observed single cells seeded much better than seeding cell fragments. Also, seeding approximately 30000 or more cells per well resulted in better patterning in comparison to seeding less than this amount; if fewer than 30000 cells are seeded they can be cultured for a time sufficient so that desired densities are achieved on the microspot(s) before switching to Matrigel-containing media. Additionally, there was no immediately apparent effect on morphology from overlaying the seeded cells with either 5% or 100% Matrigel overlays. However, a 100% Matrigel overlay may tend to obscure imaging the cultures (whether phase contrast or confocal).

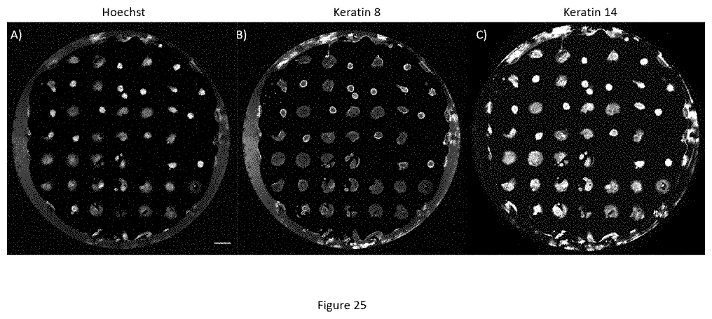

The tissue-derived adult mouse prostate aggregates were further characterized by staining with the DNA fluorochrome Hoechst 33258 and antibodies against Keratin 8, and Keratin 14 to detect the presence of luminal and basal cells, respectively (FIG. 25).

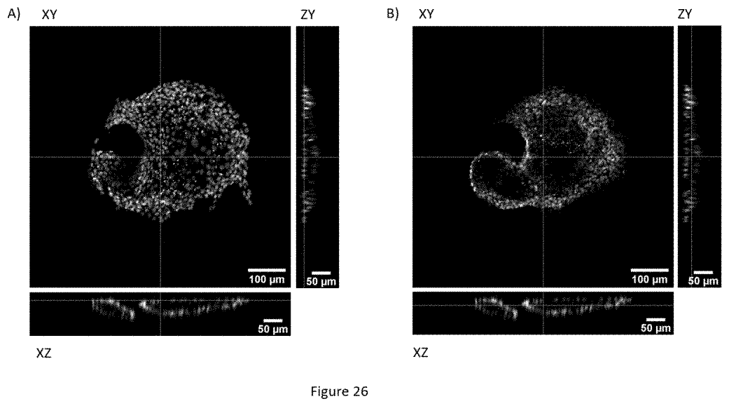

In the time course experiment set out in FIG. 12 and FIG. 13, we observed that seeded adult mouse prostate cells spontaneously formed hollow spheres, with a thickness of one cell layer. To further characterize the aggregates formed on the plurality of microspots, they were scanned using a confocal microscope. Orthogonal sections taken of an exemplary day 7 prostate aggregate formed from 55,000 cells seeded in a well of a 96-well patterned anchorage surface device and cultured with 100% Matrigel overlay that was stained with the DNA fluorochrome Hoechst 33258 clearly show the 3D structure (FIG. 26).

### Example 12

### Growing Three-Dimensional Tissue-Derived Human Pulmonary Organoids on a Coated Anchorage Surface Device

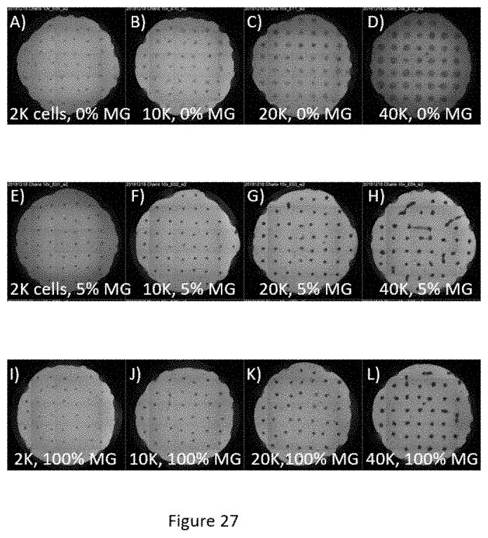

Tissue-derived human pulmonary cells (human bronchial epithelial cells) were seeded and grown for 7 days on the plurality of microspots disposed on the anchorage surface device (i.e. a 96-well patterned plate) in accordance with Examples 3 and, in the essential aspects, 5 (FIG. 27). In this embodiment, each microspot had a diameter of about 500 um and arrayed with a pitch of about 700 um.

The tissue-derived human pulmonary cells were seeded as single cells (FIG. 27A-L) in the PneumaCult™ AOK system (STEMCELL Technologies, as described in Example 14) in varying numbers: 2000 cells (A, E, I); 10000 cells (B, F, J); 20000 cells (C, G, K); and 40000 cells (D, H, L). Additionally, the effect of 0% (FIG. 27A-D) Matrigel overlay, 5% (FIG. 27 E-H) Matrigel overlay diluted in culture medium, and 100% (FIG. 27 I-L) was also tested.

Seeding approximately 10000-40000 cells per well resulted in the best patterning; if fewer than this number of cells are seeded they can be cultured for a time sufficient so that desired densities are achieved on the microspot(s) before switching to Matrigel-containing media. Additionally, there was no immediately apparent effect on morphology from overlaying the seeded cells with either 5% or 100% Matrigel overlays, but no Matrigel overlay resulted in a flat morphology. However, a 100% Matrigel overlay may tend to obscure imaging the cultures (whether phase contrast or confocal).

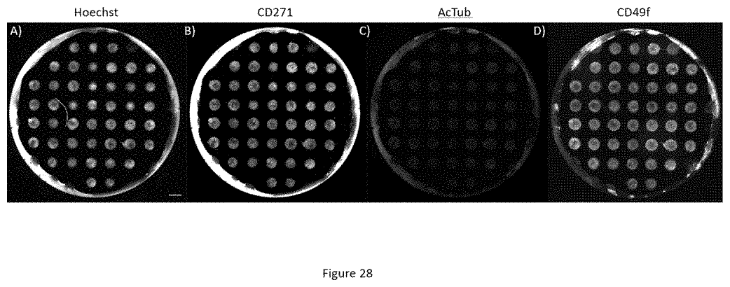

In an experiment with no Matrigel overlay (and no Matrigel added to the culture medium) day 7 tissue-derived human pulmonary aggregates (organoids) were formed from 40,000 HBECs. Such tethered aggregates were further characterized by staining with the DNA fluorochrome Hoechst 33258 and antibodies against CD271 and CD49f to detect the presence of basal cells and with antibody AcTub to confirm the absence of differentiated ciliated cells (FIG. 28).

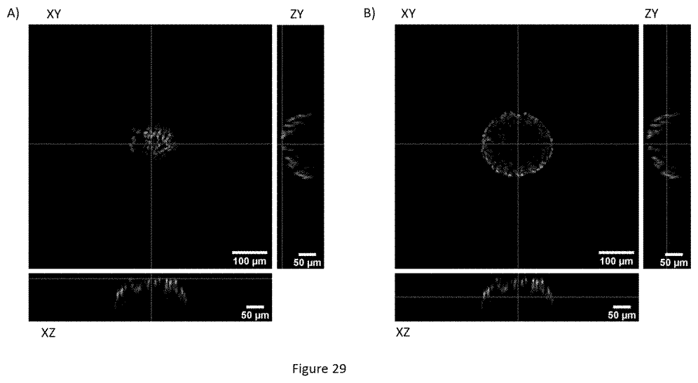

In the time course experiment set out in FIG. 12 and FIG. 13, we observed that seeded tissue-derived human pulmonary cells spontaneously formed hollow spheres, with a thickness of one cell layer. To further characterize the aggregates formed on the plurality of microspots, they were scanned using a confocal microscope. Orthogonal sections taken of an exemplary day 7 pulmonary aggregate formed from 20,000 HBECs seeded in a well of a 96-well patterned anchorage surface device and cultured with 100% Matrigel overlay that was stained with the DNA fluorochrome Hoechst 33258 clearly show the 3D structure (FIG. 29).

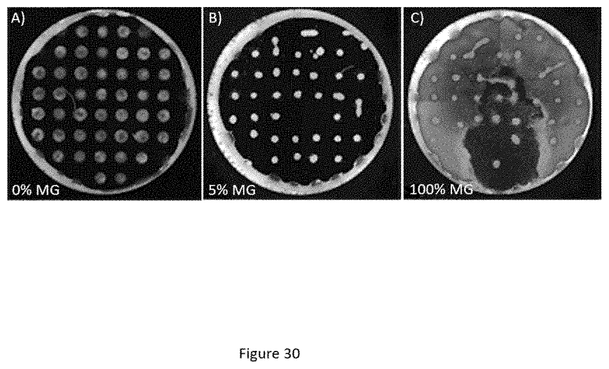

In addition to the dramatic change in morphology when even a low amount of Matrigel is added, we found that approximately 5% Matrigel diluted in culture medium, rather than 100% Matrigel overlays, permits the formation of 3D morphology while also providing for much less staining background. An example is provided in (FIG. 30), which shows a composite of Hoechst, CD271, AcTub, and CD49f staining with markedly more background staining visibly in the 100% Matrigel overlay cultures.

### Example 13

### Growing Three-Dimensional Human Pancreatic Duct Organoids from Human Pancreatic Progenitor Cells on a Coated Anchorage Surface Device

Human pancreatic progenitor cells derived from pluripotent stem cells (or PSCs) were seeded on the plurality of microspots disposed on the anchorage surface device (i.e. a 96-well patterned plate) in accordance with Examples 5 and 6. In this embodiment, each microspot had a diameter of about 500 um and arrayed with a pitch of about 700 um.

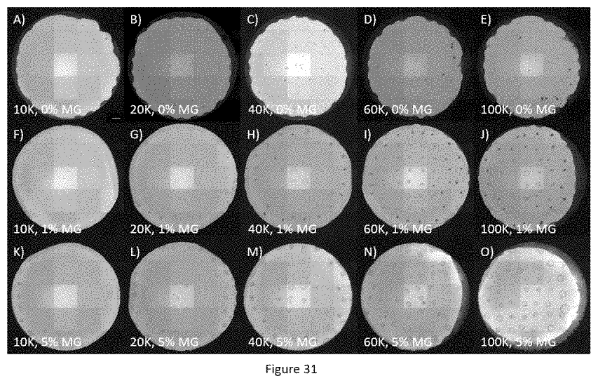

The pancreatic progenitor cells were seeded as single cells in Pancreatic Progenitor Differentiation Medium (STEMCELL Technologies) with 10 u.M ROCK inhibitor (FIG. 31A-O) in varying numbers: 10000 cells (A, F and K); 20000 cells (B, G and L); 40000 cells (C, H and M); 60000 cells (D, I and N); and 100000 cells (E, J and 0). 24 hours after seeding, the cultures were rinsed to remove unattached cells before adding fresh Pancreatic Progenitor Differentiation Medium including ROCK inhibitor. The Pancreatic Progenitor Differentiation Medium further included various dilutions of Matrigel as follows: 0% Matrigel (FIG. 31A-E); 1% Matrigel (FIGS. 31 F-.1); and 5% Matrigel (FIG. 31 K-O). After 48 hours the Pancreatic Progenitor Differentiation Medium (with ROCK inhibitor and Matrigel, as indicated) was replaced with

Pancreatic Duct Organoid Differentiation Medium 1 (STEM CELL Technologies) with ROCK inhibitor and the concentrations of Matrigel as indicated above. After an additional 48 hours of incubation the Pancreatic Duct Organoid Differentiation Medium 1 was replaced with fresh Pancreatic Duct Organoid Differentiation Medium 2 (STEMCELL Technologies) including Matrigel concentrations as indicated above but not including ROCK inhibitor. This medium was replaced every 2-3 days until fixation ahead of staining.

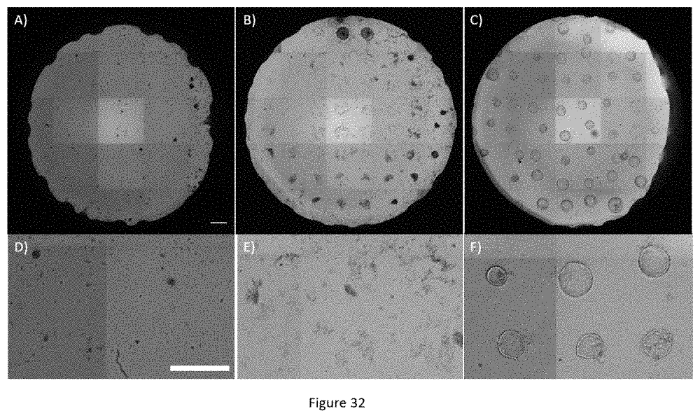

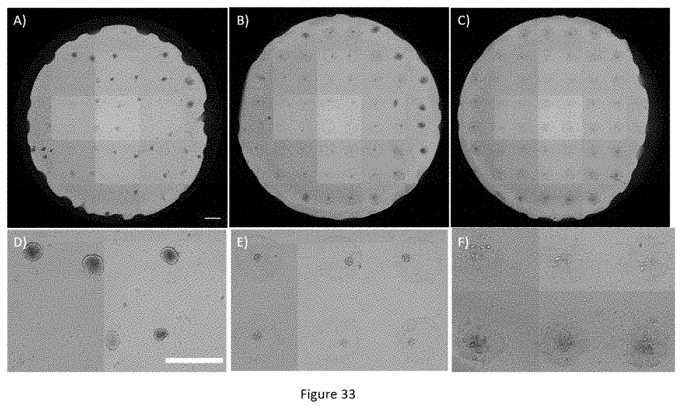

Robust sphere formation was observed at day 14 in the conditions where 60000-100000 cells were seeded and cultured (as described above) in media including 5% Matrigel. The spheres included hollow lumens with approximately the thickness of a single cell layer (FIG. 32 C and F). Cells cultured in media including 0-1% Matrigel did not survive or became washed off the anchorage surface device (FIG. 32 A, B, D and E). Notably, at day 7 the cells cultured in media including 1% Matrigel could pattern into tethered three dimensional aggregates (FIG. 33 B and E) but these cultures deteriorated by day 14 (FIG. 32 B and E). Also, at day 7 the cells cultured in media including 5% Matrigel also had acceptable patterning but were monolayer cultures (FIG. 33 C and F), indicating that sphere formation occurred between day 7 and 14.

### Example 14

### Performing a Forskolin-Induced Swelling Assay with Three-Dimensional Pulmonary Organoids from Primary Human HBECs on a Coated Anchorage Surface Device

Human bronchial epithelial cells (HBECs) from a single donor were seeded on the plurality of microspots disposed on the anchorage surface device (i.e. a 96-well patterned plate) in accordance with Examples 5 and 6. In this embodiment, each microspot had a diameter of about 500 um and arrayed with a pitch of about 700 um.

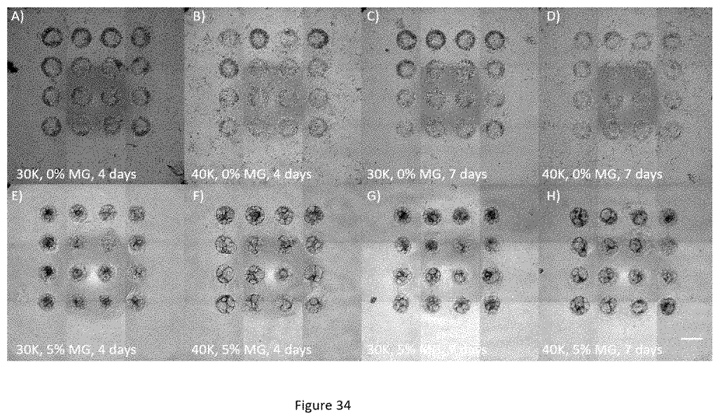

The HBECs were seeded as single cells in PneumaCult™ AOK Seeding Medium (STEMCELL Technologies) (FIG. 34A-H) in varying numbers: 30000 cells (A, C, E, and G); or 40000 cells (B, D, F, and H). 48 hours after seeding, the cultures were rinsed to remove unattached cells before adding fresh PneumaCult™ AOK Seeding Medium. The PneumaCult™ AOK Seeding Medium further included various dilutions of Matrigel as follows: 0% Matrigel (FIG. 34A-D); or 5% Matrigel (FIG. 34 E-H). Media was replaced every 48 hours. At either day 4 or day 7 after seeding, the media was changed to PneumaCult™ AOK Differentiation Medium (STEMCELL Technologies), which was replaced every 48 hours. Cultures were maintained until day 28 in PneumaCult™ AOK Differentiation Medium (STEMCELL Technologies). Note, this experiment was conducted with the presence of the antibiotic gentamicin in the media on the 96-well patterned plate.

At day 28, cells cultured in media not including Matrigel did not yield tethered three-dimensional aggregates (organoids). In contrast, cells cultured in medium including 5% Matrigel reproducibly yielded three-dimensional cystic aggregates tethered to the microspots, with each organoid having multiple lumens.

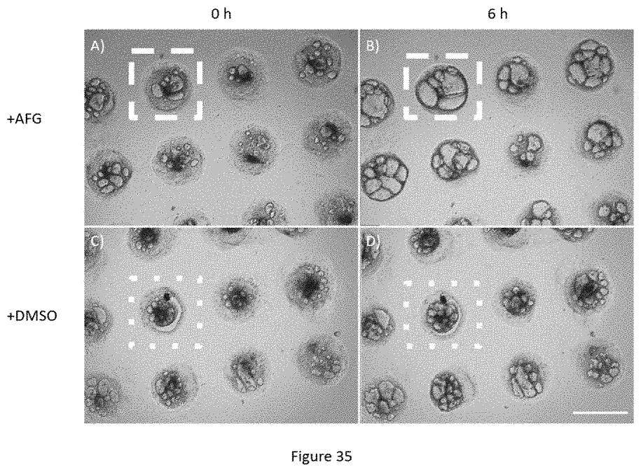

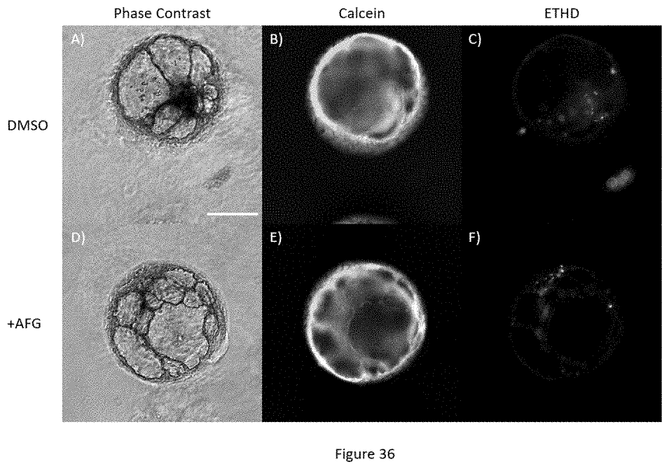

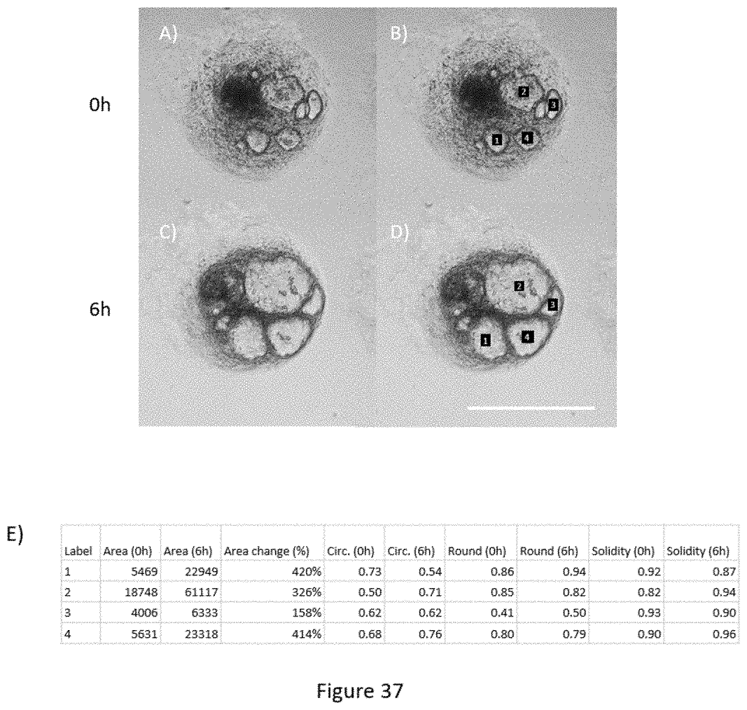

On day 28 a Forskolin-Induced Swelling (FIS) assay was performed. Cultures were imaged (T=0 hours) and then Cystic Fibrosis Transmembrane Conductance Regulators (CFTR) amiloride (20 μM), forskolin (10 μM), and genistein (25 μM) (“AFG”) or an equal volume DMSO control were added to wells as noted. After 5.5 hours Calcein AM (a live-cell viability dye) and cell-impermeant viability indicator ethidium homodimer-1 (ETHD) were added to the cultures. After 30 minutes (T=6 hours) the cultures were imaged again using phase contrast. By comparing T=0 hours (FIG. 35A) and T=6 hour (FIG. 35 B) time points the pulmonary cysts clearly swell in response to AFG. At T=6 hours, in addition to phase contrast the cultures were also fluorescently imaged to capture live and dead signals. No overt difference in live cell (Calcein) (FIG. 36 B and E) or dead cells (ETHD) (FIG. 36 C and F) was observed. High-resolution live-cell phase contrast imaging may be used to detect aggregate (organoid) lumens and to quantify their size (FIG. 37).

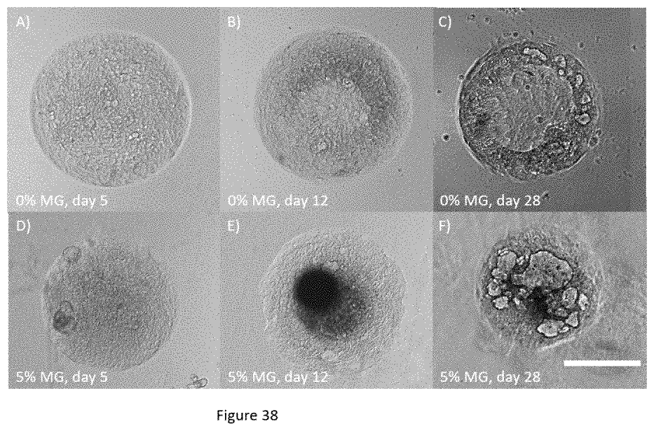

Cultures of HBECs were phase contrast imaged throughout the duration of the organoid forming assay, allowing for the development of organoids tethered on a single microspot to be tracked over time. FIG. 38 shows a representative microspot of HBECs exposed to culture medium including 0% Matrigel (FIG. 38 A-C) and a different microspot of HBECs exposed to culture medium including 5% Matrigel (FIG. 38 D-F) at day 5 (FIG. 38A and D), day 12 (FIG. 38 B and E) and day 28 (FIG. 38 C and F) after seeding. Analysis of the time course images indicates that the lumens present at day 28 were generated after day 12 and required the presence of Matrigel.

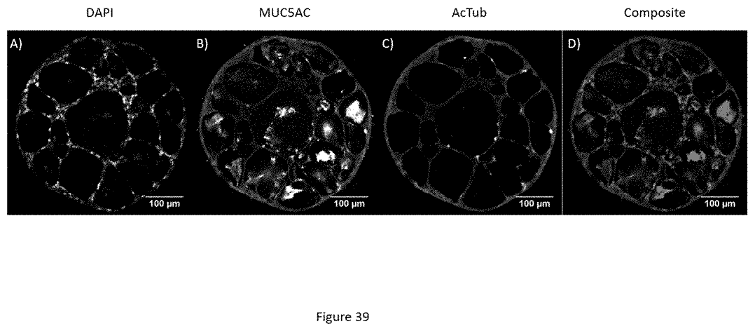

The day 28 pulmonary organoids were further characterized by staining with the DNA fluorochrome DAPI, and antibodies against Mucin 5AC (MUC5AC, (a marker of mucus) and acetylated alpha tubulin (AcTub, a cytoskeletal polymer present in cilia) (FIG. 39). The staining shows that cells cultured tethered to the anchorage surface device in pulmonary organoid promoting media yielded pulmonary organoids expressing characteristic markers; thus, the anchorage surface devices support the maturation of pulmonary organoids from seeded HBECs.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

1. A method of growing a three-dimensional aggregate of cells, the method comprising:
providing an anchorage surface having a plurality of microspots disposed on a first planar face thereof, each of the plurality of microspots
(i) having a first chemical attribute,
(ii) separated by a pitch from a microspot adjacent thereto, and
(iii) surrounded by a continuous interstitial surface having a second chemical attribute, the second chemical attribute being different than the first chemical attribute;
contacting the plurality of microspots with a cell suspension; and
culturing one or more anchorage-dependent cells of the cell suspension in a supportive culture medium under supportive culture conditions,
wherein the first chemical attribute supports growth of the one or more anchorage-dependent cells to yield the three-dimensional aggregate of cells tethered to a microspot and the second chemical attribute does not support growth of the one or more anchorage-dependent cells to yield the three-dimensional aggregate of cells.

2. The method of claim 1, further comprising contacting the plurality of microspots with a fluid coating supplement for a time sufficient to allow some or all of the fluid coating supplement to bind the plurality of microspots before contacting the plurality of microspots with the cell suspension, and optionally removing excess fluid coating supplement.

3-7. (canceled)

8. The method of claim 1, wherein the three-dimensional aggregate of cells is an embryoid body, an aggregate of undifferentiated PSC or differentiated PSC, an organoid, or a mass of chondrocytes.

9-10. (canceled)

11. The method of claim 8, wherein reduced off-target cell differentiation occurs between or about a first three-dimensional aggregate of cells and an adjacent second three-dimensional aggregate of cells compared to adjacent first and second three-dimensional aggregates of cells not grown using the anchorage surface.

12. The method of any onc of claims 8 to 11, wherein the organoid is one of: a lung organoid; a kidney organoid; a pancreatic organoid; a liver organoid; a small intestinal, including an ileal, organoid; a large intestinal, including a colonic, organoid; a stomach organoid; a prostate organoid; or a mammary organoid, and wherein the organoid is lumenized and/or poloarized.

13-15. (canceled)

16. The method of any onc of claims 1 to 15, further comprising exposing a first three-dimensional aggregate of cells to a first condition and exposing a second three-dimensional aggregate of cells to a second condition.

17-20. (canceled)

21. The method of claim 1, wherein the supportive culture medium includes a sub-gelation dilution of the fluid coating supplement.

22. The method of claim 2, wherein the fluid coating supplement is a solution of one or more extracellular matrix proteins, optionally wherein the one or more extracellular matrix proteins include one or more of fibronectin, collagen, laminin, elastin, vitronectin, entactin, heparin sulphate, proteoglycan, or Matrigel.

23-24. (canceled)

25. The method of claim 1, wherein the first chemical attribute is relatively more hydrophilic than the second chemical attribute.

26. The method of claim 1, wherein the first chemical attribute is hydrophilic and the second chemical attribute is hydrophobic, and/or the first chemical attribute carries a charge different from the second chemical attribute, and/or the first chemical attribute carries a functional group different from the second chemical attribute.

27-29. (canceled)

30. An assay using a three dimensional aggregate of cells grown in accordance with claim 1.

31. (canceled)

32. An anchorage surface for use in the method of claim 1, comprising a plurality of microspots disposed on a first planar face, each of the plurality of microspots
i) having a first chemical attribute,
ii) separated by a pitch from a microspot adjacent thereto, and
iii) surrounded by a continuous interstitial surface having a second chemical attribute, the second chemical attribute being different than the first chemical attribute,
wherein the first chemical attribute supports the tethering of biological materials thereto and the second chemical attribute is not supportive of tethering biological materials thereto.

33. The surface of claim 32, wherein the pitch is between about 20 μm and 5000 μm, and preferably between about 500 μm and 1500 μm.

34-35. (canceled)

36. The surface of claim 32, wherein a spacing between two adjacent microspots taken from a first edge of a first microspot to a closest first edge of an adjacent second microspot is between about 30 μm and 3000 μm, and preferably between about 100 μm and 1000 μm.

37. The surface of claim 32, further comprising up to 10 microspots, up to 50 microspots, up to 100 microspots, up to 250 microspots, up to 500 microspots, up to 1000 microspots, or more.

38. (canceled)

39. The surface of claim 32, further comprising a leak-proof physical barrier attached to the first face, the physical barrier circumscribing the plurality of microspots.

40. The surface of claim 39, wherein the physical barrier defines at least one side wall and an open top end.

41. The surface of claim 39, further comprising more than one physical barrier attached to the first face, each of the more than one physical barrier circumscribing each of the plurality of microspots or a respective set of plurality of m icrospots.

42-47. (canceled)

48. The surface of claim 32, wherein the first chemical attribute is relatively more hydrophilic than the second chemical attribute.

49. The surface of claim 32, wherein the first chemical attribute is hydrophilic and the second chemical attribute is hydrophobic, and/or the first chemical attribute carries a charge different from the second chemical attribute, and/or the first chemical attribute carries a functional group different from the second chemical attribute.

50-54. (canceled)
